(19) United States
(12) Patent Application Publication
(10) Pub. No.: US 2021/0353619 A1
(43) Pub. Date: Nov. 18, 2021

(54) TREATMENT FOR VENETOCLAX-RESISTANT AND VENETOCLAX-SENSITIVE ACUTE MYELOID LEUKEMIA

(71) Applicant: OREGON HEALTH & SCIENCE UNIVERSITY, Portland, OR (US)

(72) Inventors: Jeffrey W. Tyner, Portland, OR (US); Haijiao Zhang, Portland, OR (US)

(21) Appl. No.: 17/290,649

(22) PCT Filed: Oct. 30, 2019
(86) PCT No.: PCT/US2019/058923

§ 371 Date: Apr. 30, 2021

Related U.S. Application Data
(60) Provisional application No. 62/774,087, filed on Nov. 30, 2018.
(60) Provisional application No. 62/754,386, filed on Nov. 1, 2018.

(51) Int. Cl. A61K 31/496; A61K 45/06; A61K 31/519; A61K 31/44; A61K 31/5355; A61K 31/5377; A61P 35/02

(52) U.S. Cl. CPC A61K 31/496; A61K 45/06; A61K 31/519; A61P 35/02; A61K 31/5355; A61K 31/5377; A61K 31/44

(57) ABSTRACT

Provided herein are methods and therapeutic combinations useful in the treatment of venetoclax-resistant Acute Myeloid Leukemia and of venetoclax-sensitive Acute Myeloid Leukemia.

### GOVERNMENT SUPPORT

This invention was made with government support under U01 CA217862 and U54 CA224019 awarded by the NIH. The government has certain rights in the invention.”

### FIELD OF THE INVENTION

The present invention concerns methods of identifying and treating venetoclax-resistant Acute Myeloid Leukemia (AML).

### BACKGROUND OF THE INVENTION

BCL2 is an antiapoptotic protein commonly expressed in hematologic malignancies. Overexpression of BCL-2 is a poor prognostic factor in acute myeloid leukemia (AML). Venetoclax (ABT-199) is a highly selective BCL2 inhibitor that can induce cell death in multiple leukemia cell lines. Recently, venetoclax received an FDA breakthrough therapy designation for use in combination with hypomethylating agents in treatment-naive patients with AML who are unfit for intensive chemotherapy. However, venetoclax was only modestly effective as monotherapy in relapsed/refractory AML (19% CR/CRi).

Acute myeloid leukemia (AML) is a molecularly and clinically heterogeneous disease with poor prognosis¹⁻³. Despite substantial research, AML treatment did not evolve profoundly until recently. Since 2017, the US Food and Drug Administration (FDA) approved several new targeted agents for the treatment of AML, including the BCL2-inhibitor venetoclax⁴⁻¹⁰.

AML cells often up-regulate pro-survival members of the BCL2 protein family, such as BCL2 and MCL1, to avoid apoptosis^{11,12}. Overexpression of BCL2 is implicated in sustaining survival of AML cells, conferring a poor prognosis, and inducing treatment resistance^{12,13}. Therefore, targeting BCL2 has long been an attractive strategy to treat AML and other hematological malignancies. Among others, venetoclax is a potent BCL2-selective BH3-mimetic that induces responses in the majority of patients with previously treated Chronic lymphocytic leukemia (CLL) or small lymphocytic lymphoma, and therefore was approved by FDA for the treatment of these diseases^{14,15}. For de novo older AML patients, venetoclax in combination with low dose hypomethylation agents induced about 70% response rates^{6-8,16}. However, it was only modestly effective in relapsed/refractory and/or secondary AML as monotherapy (19% complete remission (CR)/complete remission with incomplete blood count recovery (CRi)) or coupled with hypomethylation treatment (54% CR/CRi)^{4,9}. Moreover, similar to other targeted therapy, subsequently acquired resistance was observed in most patients following venetoclax treatment.

Several mechanisms have been shown to contribute to venetoclax resistance. One of the main determinants of resistance to venetoclax is up-regulation of other anti-apoptotic BCL2 family proteins, including MCL1, BCL2L1 (BCL-xL), and BCL2L2 (BCL-w)¹⁷⁻²⁰. Another major factor affecting leukemia cell survival and drug sensitivity is disruption of mitochondrial structure and metabolic-related pathways²¹⁻²⁵. Recent genome-wide CRISPR screening studies have identified that knockout of TP53, BAX, and PMAIP1 confers venetoclax resistance, and depletion of mitochondrial chaperonin CLPB can sensitize AML to venetoclax^{24,26}. Studies have also shown that venetoclax-resistant cells exhibit increased phospho-ERK (pERK) and pAKT, suggesting that upregulation of MAPK and AKT pathways may lead to venetoclax resistance^{20,27,28}. However, most of these studies were conducted in AML cell lines, which do not recapitulate the clinical diversity and genetic heterogeneity of primary AML samples. We, therefore, integrated clinical parameters, whole exome sequence (WES) data, and RNAseq gene expression data with primary AML sample venetoclax screening data from the Beat AML cohort²⁹, to identify more clinically relevant phenotypic and genomic determinants as biomarkers to predict response to venetoclax.

There remains a need to identify and verify biomarkers to predict venetoclax sensitivity and resistance in AML, and to identify potential venetoclax combination treatment strategies.

### SUMMARY OF THE INVENTION

We investigated approximately 200 primary patient samples of AML and correlated clinical parameters, whole exome sequence data, and RNAseq gene expression data with in vitro drug screening data (drug area under the curve (AUC)) to identify subsets of AML samples with sensitivity or resistance to venetoclax alone and in combinations with small molecular inhibitors (including Array-382, dasatinib, doramapimod (BIRB 796), quizartinib, JQ-1, idelalisib, quizartinib, palbociclib, panobinostat, ruxolitinib, sorafenib, and trametinib).

Provided herein are methods and therapeutic combinations useful in the treatment of venetoclax-resistant Acute Myeloid Leukemia and of venetoclax-sensitive Acute Myeloid Leukemia.

### BRIEF DESCRIPTION OF THE DRAWINGS

FIG. 1 provides Pearson correlation plots showing that gene expression of BCL2A, MCL1, CD14, and CLEC7A positively correlates with venetoclax AUC, and BCL2 expression negatively correlates with venetoclax AUC.

FIG. 2 graphs the mean±SEM of venetoclax AUCs of primary AML patient samples in the presence or absence of CD14 expression detected by flow cytometry.

FIG. 3 demonstrates higher venetoclax AUCs in AML samples with PTPN11 mutations compared wild type

FIG. 4 graphs higher MCL1 expression in samples harboring PTPN11 mutations.

FIG. 5 depicts the mean±SEM of cell viabilities of inducible PTPN11 WT or A72D mutant transduced cells in the presence of venetoclax with or without doxycycline.

FIG. 6 depicts the mean±SEM of cell viabilities of inducible PTPN11 WT or A72D mutant transduced cells in the presence of idasanutlin with or without doxycycline.

FIG. 7 demonstrate higher venetoclax AUCs in AML samples with KRAS mutations vs KRAS WT.

FIG. 8 depicts higher BCL2A1 expression in samples harboring KRAS mutations.

FIG. 9 provides a Pearson correlation plot of a negative correlation between BCL2A1 expression and venetoclax-palbociclib AUC.

FIG. 10(a) represents cell viabilities of an inducible PTPN11 WT or A72D mutant transduced cells in the presence of AZD5991, with or without doxycycline

FIG. 10(b) represents cell viabilities of the PTPN11 A72D mutant induced by Doxycycline, in the presence of AZD5991, venetoclax, or a combination of both agents.

FIG. 11 depicts higher BCL2A1 expression in samples harboring SF3B1 mutations.

FIG. 12 demonstrates higher venetoclax AUCs in AML samples with SF3B1 mutations compared to samples with wild type (WT) SF3B1.

FIG. 13A provides a comparison of Venetoclax AUC among different chromosome translocation groups. The presence/absence of translocations was determined from karyotype. Only translocations that were found in ≥3 patients were considered.

FIG. 13B provides a comparison of Venetoclax AUC among different common AML mutation groups. Mutational data were collected by either targeted sequencing, whole-exome sequencing, or targeted polymerase chain reaction (PCR)-based methods (FLT3-ITD and NPM1).

FIG. 13C provides Venn diagrams depicting distribution and overlapping of the three groups of genes: brown cluster genes, most correlated single genes, and top 20% differentially expressed genes. Notably, we did not perform a familywise error correction.

FIG. 13D graphs is the Reactome pathway hierarchy containing all pathways significant in at least 2 of the 3 analyses (WGCNA brown gene expression cluster; single genes correlated with venetoclax AUC with r>=0.5 or r<=0.5; and the most differentially expressed genes between the top 20% and the bottom 20% AUC samples.) All pathways were assessed for significance at the FDR<0.05 level. Black indicates significance; whereas grey indicates non-significance. The pathway names are colored by the magnitude of significance with blue being most significant and light blue being less significant in terms of the maximum significant FDR. TLR: Toll-like receptor.

FIG. 14A provides data representing −log 10(FDR) values vs the Pearson r values between venetoclax AUC and BCL2 family gene expression levels from AML patient samples, determined by the Pearson correlation coefficients.

FIG. 14B represents the correlation between BCL2A1 gene expression levels and venetoclax AUC (n=186) from Beat AML patient samples determined by the Pearson correlation coefficient: ****; p<0.0001.

FIG. 14C represents the correlation between BCL2A1 and BCL2 gene expression levels from Beat AML patient samples (n=186) determined by the Pearson correlation coefficient and expressed as **** p<0.0001.

FIG. 14D depicts a Western blot showing knockdown of BCL2A1 from Molm13 and MV4-11 cells transduced with Dox-inducible BCL2A1 virus in the presence of Dox. Actin was used as a control. Blot shown is representative of two independent experiments with consistent results.

FIG. 14E provides representative graphs depicting higher mean±SEM of cell viabilities (three technical replicates) of Molm13 and MV4-11 cells transduced with Dox-inducible BCL2A1 virus in the presence of Dox and dose gradients of venetoclax (top). Bar graphs depict higher mean±SEM of venetoclax IC50 (five biological replicates) of Molm13 and MV4-11 cells transduced with Dox-inducible BCL2A1 virus in the presence of Dox (bottom). Significance was determined using Mann-Whitney tests and expressed as * p<0.05.

FIG. 14F depicts Western blot analysis of BCL2 family proteins and full-length/cleaved PARP of Molm13 cells transduced with Dox-inducible BCL2A1 virus in the absence or presence of Dox and dose gradients of venetoclax for 12 h. Actin was used as a control. Blots shown are representative of two independent experiments with consistent results.

FIG. 14G provides graphs depicting the mean±SEM of cell viabilities of Molm13 and MV4-11 cells transduced with Dox-inducible BCL2A1 virus in the absence or presence of Dox and indicated venetoclax combination and other BCL2 inhibitors. Graphs shown are representative of two independent experiments.

FIG. 14H provides graphs depicting the correlation between BCL2A1 gene expression levels from AML patient samples and ABT-737 (n=233), AZD4320 (n=119), or AZD5991 (n=178) with r and p values summarized on the right bottom determined by the Pearson correlation coefficient tests.

FIG. 14I provides Western blot analyses of BCL2 family proteins in AML cell lines. Vinculin was used as controls.

FIG. 14J provides a graph depicting the mean±SEM of cell viabilities (three technical replicates) of AML cell lines in the presence of dose gradients of venetoclax. The graph is representative of two independent experiments.

FIG. 14K depicts a Western blot analysis of BCL2A1 expression of U937 cells transduced with control shS or shRNA targeting BCL2A1 (sh1, sh2, and sh3UTR). Actin was used as a control.

FIG. 14L provides a graph depicts mean±SEM of percentages of viable (Annexin V−/PI−), apoptotic (Annexin V+/PI−) and necrotic cells (Annexin V+/PI+) of U937 cells transduced with shS, sh1, and sh2 48 h after FACS sorting as assessed by flow cytometry. Significance was determined using Kruskal-Wallis tests comparing to the respective shS control and expressed as * p<0.05 and ** p<0.01.

FIG. 14M provides a graph depicting the mean±SEM of percentage changes of indicated double transduced cells (GFP+) after 72 h cell culture. Significance was determined using Mann-Whitney tests, comparing to the respective shS control and expressed as * p<0.05, ** p<0.01, and ***<0.001.

FIG. 14N provides a graph depicting the mean±SEM of percentage changes of shS or sh3UTR transduced (GFP+) U937 cells expressing Dox-inducible BCL2A1 construct in the presence or absence of Dox after 72 h of cell culture. Significance was determined using Mann-Whitney tests comparing to the respective Dox-control and expressed as * p<0.05.

FIG. 14O provides a graph depicting the mean±SEM of percentage changes of shS and sh1 transduced (GFP+) primary AML cells (left) and CD34+ cord blood HSPCs (right). Statistical significances were determined using Kruskal-Wallis tests comparing to the respective shS control and expressed as ** p<0.01 and ****<0.0001.

FIG. 14P provides a graph depicting the mean±SEM of cell viabilities (three technical replicates) of Molm13 cells transduced with shS, sh1, and Sh3UTR in the presence of dose gradients of venetoclax. The graph is representative of two independent experiments.

FIG. 14Q provides a graph depicts the mean±SEM of the percentage of shS and sh1 transduced (GFP+) primary AML cells (left) and CD34+ cord blood HSPCs (right) in the presence of concentration gradients of venetoclax. Statistical significances were determined using Kruskal-Wallis tests in comparison to the respective shS control and expressed as ** p<0.01 and ****<0.0001.

FIG. 15A represents −log 10(FDR) values vs the Pearson r values between venetoclax AUC and gene expression levels of cell surface GO term genes from AML patient samples, determined by the Pearson correlation coefficient.

FIG. 15B provides correlation between CD369 and CD14 gene expression levels and venetoclax AUC (n=189) from AML patient samples determined by the Pearson correlation coefficient: ****; p<0.0001.

FIG. 15C provides a graph depicting the mean±SEM of venetoclax AUCs of primary AML patient samples categorized based on the presence or absence of CD14 expression detected by clinical immunophenotyping.

FIG. 15D provides a graph depicts the mean±SEM of venetoclax AUCs of primary AML patient samples in the non-M4/M5 and M4/M5 groups for those samples with clinical annotation. Significance was determined using Mann-Whitney tests and expressed as ** p<0.05.

FIG. 15E provides graphs depicting the mean±SEM of cell viabilities (three technical replicates) of CD369/CD14+ or CD369/CD14− primary leukemia blast cells in the presence of dose gradients of venetoclax.

FIG. 15F provides a graph depicting the mean±SEM of ΔΔCt of indicated apoptosis-related genes between CD369 and/or CD14 positive and negative primary AML blasts (four independent samples). Actin and GAPDH were used as controls. Significance was determined using Mann-Whitney tests and expressed as * p<0.05.

FIG. 15F provides a Graph depicting positive correlations between CD369 and BCL2A1 gene expression of primary patient samples from Beat AML (n=601), TCGA AML (n=173), CML (n=102), and CNL/aCML/CMML/MDS/MPN-U (n=94) cohort determined by the Pearson correlation coefficient: **** p<0.0001.

FIG. 16A provides a graph demonstrating higher venetoclax AUCs in AML samples with KRAS mutations compared with KRAS WT samples. Significance was determined using Mann-Whitney tests and expressed as ** p<0.05.

FIG. 16B provides a table summarizing the change of mutation VAF and venetoclax AUC from an AML patient at diagnosis and disease relapse.

FIG. 16C presents a graph depicting the mean±SEM of colony numbers of KRAS G12D transduced BM HSPCs treated with gradient concentrations of venetoclax or trametinib. Statistical significance was determined using Kruskal-Wallis tests comparing each group to the non-treated group and expressed as* p<0.05. The graph is representative of two independent experiments.

FIG. 16D provides representative graphs depicting the mean±SEM of cell viabilities (three technical replicates) of Molm13 transduced with Dox-inducible KRAS WT and G12D virus in the absence or presence of Dox (top) and dose gradients of venetoclax. The graph depicts the mean±SEM of IC50 (8 biological replicates) of Molm13 cells expressing Dox-inducible KRAS WT and G12D virus in the absence or presence of Dox (bottom). Significance was determined using a Kruskal-Wallis test and expressed as * p<0.05.

FIG. 16E presents graphs depicting the mean±SEM of cell viabilities (three technical replicates) of Molm13 transduced with Dox-inducible KRAS WT and G12D virus in the absence or presence of Dox and dose gradients of venetoclax combinations and other BCL2 family inhibitors. The graph is representative of two independent experiments.

FIG. 16F provides Western blot analyses of BCL2 family proteins extracted from Molm13 cells transduced with Dox-inducible KRAS WT and G12D virus in the absence or presence of Dox (Left). BAX expression in Molm13 cells transduced with empty vector, KRAS WT, and KRAS G12D constitutive lentivirus (right).

FIG. 16G provides Western blot analyses of BCL2 family proteins and full-length/cleaved PARP of Molm13 cells transduced with Dox-inducible KRAS WT and G12D virus in the absence or presence of Dox and dose gradients of venetoclax for 12 h. Vinculin was used as a control. Blots are representative of three independent experiments.

FIG. 17A presents a graph demonstrating higher venetoclax AUCs in AML samples with PTPN11 mutations compared with PTPN11 WT samples. Significant was determined using a Mann-Whitney test and expressed as ** p<0.05.

FIG. 17B presents a graph depicting the mean±SEM of colony numbers for three replicates of PTPN11 A72D transduced BM HSPCs treated with gradient concentrations of venetoclax or trametinib. Statistical significance was determined using Mann-Whitney tests comparing each group to the non-treated group and expressed as* p<0.05. The graph is representative of two independent experiments.

FIG. 17C presents Western blot analyses of PTPN11 extracted from HEKT293 cells transduced with Dox-inducible PTPN11 WT and A72D virus in the absence or presence of Dox. Actin was used as a control.

FIG. 17D provides representative graphs depicting the mean±SEM of cell viabilities (three technical replicates) of Molm13 transduced with Dox-inducible PTPN11 WT and A72D virus in the absence or presence of Dox and dose gradients of venetoclax (top). Bar graph depicts the mean±SEM of venetoclax IC50 (six biological replicates) of the above experiment (bottom). Significance was determined using a Kruskal-Wallis test and expressed as * p<0.05.

FIG. 17E presents representative graphs depicting the mean±SEM of cell viabilities (three technical replicates) of Molm13 transduced with Dox-inducible PTPN11 WT and A72D virus in the absence or presence of Dox (top) and dose gradients of venetoclax combinations and other BCL2 inhibitors. The graph is representative of two independent experiments.

FIG. 17F and FIG. 17G present Western blot analyses of BCL2 family proteins of Molm13 cells transduced with Dox-inducible PTPN11 WT and A72D virus in the presence of Dox and dose gradients of venetoclax for 12 h. Actin was used as a control. Blots are representative of three independent experiments.

FIG. 18A provides graphs depicting the mean±SEM of cell viabilities (three technical replicates) of indicated cells in the absence or presence of Dox and dose gradients of venetoclax, AZD5991, and two drugs in combination. The graphs are representative of two independent experiments.

FIG. 18B is a graph depicting the AUCs of the indicated drug from 5 primary AML samples.

FIG. 18C provides a graph depicting the mean±SEM of drug AUCs of venetoclax, AZD5991, and two drugs in combination from 10 primary AML samples. Significance was assessed using a Kruskal-Wallis test and expressed as **** p<0.0001.

FIG. 18D schematically illustrates the in vivo model of BCL2A1 mediated venetoclax resistance (MV4-11 cells overexpressing BCL2A1).

FIG. 18E is a graph depicting mean±SEM of luciferase intensity (average radiance (p/s/cm2/sr) from three biological replicates) of MV4-11 cells expressing BCL2A1 in the presence of indicated treatment. Significance was assessed using a Kruskal-Wallis test at each individual time point and expressed as * p<0.05 (Venetoclax vs. Venetoclax/AZD5991).

FIG. 18F presents survival curves of mice transplanted with BCL2A1 and luciferase-GFP treated with indicated drugs. P values were calculated using a log-rank (Mantel-Cox) test.

FIG. 18G provides a graph depicting the mean±SEM of BM engraftment (three biological replicates) of SU176 patient AML cells in NSG mouse before and during treatment. Significance was assessed using a Kruskal-Wallis test at each individual time point.

FIG. 18H presents survival curves of mice transplanted with blasts from SU176 leukemia sample harboring a KRAS G12D mutation treated with indicated drugs. P values were calculated using a log-rank (Mantel-Cox) test.

FIG. 18I represents Western blot analyses of BCL2 family proteins of Molm13 cells expressing BCL2A1 (left) and PTPN11 A72D (right) in the presence of Dox-treated with dose gradients of venetoclax, AZD5991 and two drugs in combination for 12 h. Blots are representative of two independent experiments.

FIG. 18J provides a graph depicting mean±SEM of cell viabilities (three technical replicates) of Molm13 and MV4-11 cells transduced with sgRNA/Cas9 control (sgCtrl) or sgRNA/Cas9 targeting BAX (sgBAX) treated with each concentration of venetoclax and AZD5991 in combination. The graph is representative of two independent experiments.

FIG. 18K provides a graph depicting mean±SEM of cell viabilities (three technical replicates) of Molm13 cells expressing Dox-inducible BCL2A1 and co-transduced with sgCtrl or sgBAX treated with venetoclax and AZD5991 in combination in the presence or absence of Dox. The graph is representative of two independent experiments.

FIG. 19A provides a graph depicting the 95% Cl and Hodges-Lehmann median difference of venetoclax AUC in the presence or absence of mutations in the indicated gene calculated using Mann Whitney tests.

FIG. 19B provides a graph depicting ABT-737 AUC compared among different common AML mutation groups. Significance was determined using Kruskal-Wallis tests, and corrected for multiple comparisons (Bonferroni correction), and expressed as #<0.05 before Bonferroni correction, however,>0.05 after Bonferroni correction.

FIG. 19C provides a graph depicting the 95% Cl and Hodges-Lehmann median difference of ABT-737 AUC in the presence or absence of mutations in the indicated gene calculated using Mann-Whitney tests.

FIG. 19D provides a heatmap depicting distributions of TP53, SF3B1, PTPN11 and KRAS mutations in BeatAML cohort. Each column displays each patient; each row denotes a specific gene. The mutation VAF is colored.

FIG. 20A provides graphs depicting the mean±SEM of cell viabilities of Molm13 cells transduced with Dox-inducible BCL2A1 virus in the absence or presence of Dox and indicated inhibitors. The graph is representative of two independent experiments.

FIG. 20B provides data representing −log 10(p) values vs the person r values between ABT-737 AUC and BCL2 family gene expression RPKM levels from AML patient samples, determined by the person correlation coefficients.

FIG. 20C provides graphs depicting the correlation between BCL2A1 gene expression levels and indicated inhibitor AUC (n=186) from BeatAML patient samples determined by the person correlation coefficient: **; p<0.01 and ****; p<0.0001.

FIG. 20D provides graphs of a comparison of expression of BCL2, BCL2A1, and MCL1 among different chromosome translocation groups. Significance was determined using Kruskal-Wallis tests and expressed as * p<0.05 and ** p<0.01.

FIG. 20E provides graphs depicting the correlation between BCL2A1 gene expression levels and BM or PB blast percentage from BeatAML patient samples determined by the person correlation coefficient: ****; p<0.0001.

FIG. 20F provides a graphs depicting the BCL2A1 gene expression levels in transferred or not transformed AML samples from the BeatAML cohort. Significance was determined using Mann-Whitney tests and expressed as **** p<0.0001.

FIG. 20G provides a graph representing the expression of BCL2A1 compared among different AML FAB subgroups. Significance was determined using Kruskal-Wallis tests and expressed as * p<0.05, ** p<0.01, and ****; p<0.0001.

FIG. 21 provides graphs representing the expression of CD369 and CD14 was compared among different chromosome translocation and FAB groups. Significance was determined using Kruskal-Wallis tests and expressed as * p<0.05, ** p<0.01, ***p<0.001, and ****; p<0.0001.

FIG. 22A provides graphs depicting the mean±SEM of cell viabilities (three replicates) of CTS or MV4-11 transduced with Dox-inducible KRAS WT and G12D virus in the absence or presence of Dox (top) and dose gradients of venetoclax. The graph is representative of two independent experiments.

FIG. 22B provides a graph demonstrating similar venetoclax AUCs in AML samples with NRAS mutations compared with NRAS WT samples. Significant was determined using Mann-Whitney tests.

FIG. 22C provides representative graphs depicting similar mean±SEM of cell viabilities of NRAS WT or G12D transduced cells in the presence of dose gradients of venetoclax. The graph is representative of two independent experiments.

FIG. 22D presents a graph demonstrating higher ABT-737 AUCs in AML samples with KRAS mutations compared with KRAS WT samples. Significant was determined using Mann-Whitney tests and expressed as *,0.05.

FIG. 22E presents representative graphs depicting similar mean±SEM of cell viabilities (three technical replicates) KRAS WT or G12D transduced cells in the presence of dose gradients of Azacytidine or Cytarabine. The graph is representative of two independent experiments.

FIG. 23A provides a graph depicting higher mean±SEM of cell viabilities (three technical replicates) of PTPN11 G12D transduced cells in the presence of Dox.

FIG. 23B provides a graph depicting the mean±SEM of IC50 of Molm13 cells expressing PTPN11 G12D transduced cells in the presence of Dox. Significance was determined using a Kruskal-Wallis test and expressed as * p<0.05.

FIG. 23C provides a graph depicting similar mean±SEM of cell viabilities of PTPN11 WT or A72D transduced CTS cells in the presence dose gradients of venetoclax. The graph is representative of two independent experiments.

FIG. 23D provides a graph demonstrating lower AZD5991 AUCs in AML samples with PTPN11 mutations compared with PTPN11 WT samples. Significant was determined using Mann-Whitney tests and expressed as *,0.05.

FIG. 24A provides graphs depicting mean±SEM of cell viabilities of BCL2A1, and KRAS/PTPN11 WT and mutant transduced cells treated with indicated inhibitors in the presence or absence of Dox. Excess over Bliss (EOB) was used to calculate the expected effect of the combination.

FIG. 24B provides graphs depicting mean±SEM of cell viabilities Stanford primary leukemia samples treated with indicated inhibitors. EOB was used to calculate the expected effect of the combination.

FIG. 24C provides graphs depicting mean±SEM of cell viabilities Stanford primary leukemia samples treated with inhibitors. EOB was used to calculate the expected effect of the combination.

FIG. 24D depicts a Western blot showing knockout of BAX expression by CRISPR targeting BAX, but not Ctrl CRISPR from both Molm13 and MV4-11 cells.

FIG. 24E provides graphs depicting the mean±SEM of cell viabilities (three technical replicates) of indicated cells in the presence or absence of Dox or indicated inhibitor. The graph is representative of two independent experiments.

### DETAILED DESCRIPTION OF THE INVENTION

Provided herein are methods of treating venetoclax-resistant and venetoclax-sensitive Acute Myeloid Leukemia in a human subject.

### Venetoclax-Resistant AML

Provided is a method of diagnosing and treating venetoclax-resistant Acute Myeloid Leukemia in a human subject, the method comprising:
a) obtaining a biological sample from the human subject;
b) detecting whether a high level of expression of CLEC7A (CD369), BCL2A1, or both of CLEC7A (CD369) and BCL2A1 is present in the biological sample;
c) diagnosing the human subject with venetoclax-resistant Acute Myeloid Leukemia when the presence of a high level of expression of CLEC7A (CD369), BCL2A1, or both of CLEC7A (CD369) and BCL2A1 is detected in the biological sample; and
d) administering to the human subject in need thereof:
   i) a pharmaceutically effective amount of venetoclax, or a pharmaceutically acceptable salt thereof; and
   ii) a pharmaceutically effective amount of palbociclib, or a pharmaceutically acceptable salt thereof.

In the methods herein, a biological sample taken from the human subject may be a blood or bone marrow sample.

Also provided is a method of diagnosing and treating venetoclax-resistant Acute Myeloid Leukemia in a human subject, the method comprising:
a) obtaining a biological sample from the human subject;
b) detecting whether a high level of expression of CLEC7A (CD369), BCL2A1, or both of CLEC7A (CD369) and BCL2A1 is present in the biological sample;
c) diagnosing the human subject with venetoclax-resistant Acute Myeloid Leukemia when the presence of a high level of expression of CLEC7A (CD369), BCL2A1, or both of CLEC7A (CD369) and BCL2A1 is detected in the biological sample; and
d) administering to the human subject in need thereof:
   i) a pharmaceutically effective amount of venetoclax, or a pharmaceutically acceptable salt thereof; and
   ii) a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

Also provided is a method of treating venetoclax-resistant Acute Myeloid Leukemia in a patient, the method comprising administering to the patient:
i) a pharmaceutically effective amount of venetoclax, or a pharmaceutically acceptable salt thereof; and
ii) a pharmaceutically effective amount of palbociclib, or a pharmaceutically acceptable salt thereof.

Also provided is a method of treating venetoclax-resistant Acute Myeloid Leukemia in a patient, the method comprising administering to the patient:
i) a pharmaceutically effective amount of venetoclax, or a pharmaceutically acceptable salt thereof; and
ii) a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether one or more mutations selected from the group of a TET2 mutation, a KRAS mutation, a PTPN11 mutation, and a SF3B1 mutation is present in the biological sample;
c) diagnosing the human subject with venetoclax-resistant Acute Myeloid Leukemia when the presence of one or more mutations selected from the group of a TET2 mutation, a KRAS mutation, a PTPN11 mutation, and a SF3B1 mutation is detected in the biological sample; and
d) administering to the human subject in need thereof a pharmaceutically effective amount of venetoclax, or a pharmaceutically acceptable salt thereof, and a pharmaceutically effective amount of palbociclib, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether one or more mutations selected from the group of a TET2 mutation, a KRAS mutation, a PTPN11 mutation, and a SF3B1 mutation is present in the biological sample;
c) diagnosing the human subject with venetoclax-resistant Acute Myeloid Leukemia when the presence of one or more mutations selected from the group of a TET2 mutation, a KRAS mutation, a PTPN11 mutation, and a SF3B1 mutation is detected in the biological sample; and
d) administering to the human subject in need thereof:
   iii) a pharmaceutically effective amount of venetoclax, or a pharmaceutically acceptable salt thereof; and
   iv) a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether one or more mutations selected from the group of a TET2 mutation, a KRAS mutation, a PTPN11 mutation, and a SF3B1 mutation is present in the biological sample;
c) detecting whether a high level of expression of CLEC7A (CD369), BCL2A1, or both of CLEC7A (CD369) and BCL2A1 is present in the biological sample;
d) diagnosing the human subject with venetoclax-resistant Acute Myeloid Leukemia when the presence of one or more mutations selected from the group of a TET2 mutation, a KRAS mutation, a PTPN11 mutation, and a SF3B1 mutation and/or a high level of expression of CLEC7A (CD369), BCL2A1, or both of CLEC7A (CD369) and BCL2A1 is detected in the biological sample; and
e) administering to the human subject in need thereof:
f) a pharmaceutically effective amount of venetoclax, or a pharmaceutically acceptable salt thereof; and
g) a pharmaceutically effective amount of palbociclib, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether one or more mutations selected from the group of a TET2 mutation, a KRAS mutation, a PTPN11 mutation, and a SF3B1 mutation is present in the biological sample;
c) detecting whether a high level of expression of CLEC7A (CD369), BCL2A1, or both of CLEC7A (CD369) and BCL2A1 is present in the biological sample
d) diagnosing the human subject with venetoclax-resistant Acute Myeloid Leukemia when the presence of one or more mutations selected from the group of a TET2 mutation, a KRAS mutation, a PTPN11 mutation, and a SF3B1 mutation and/or a high level of expression of CLEC7A (CD369), BCL2A1, or both of CLEC7A (CD369) and BCL2A1 is detected in the biological sample; and
e) administering to the human subject in need thereof:
f) a pharmaceutically effective amount of venetoclax, or a pharmaceutically acceptable salt thereof; and
g) a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

Provided is a method of diagnosing and treating venetoclax-resistant Acute Myeloid Leukemia in a human subject wherein a PTPN11 mutation is present, the method comprising:
a) obtaining a biological sample from the human subject;
b) detecting whether PTPN11 mutation is present in the biological sample;
c) diagnosing the human subject with venetoclax-resistant Acute Myeloid Leukemia when a PTPN11 mutation is detected in the biological sample; and
d) administering to the human subject in need thereof:
e) a pharmaceutically effective amount of venetoclax, or a pharmaceutically acceptable salt thereof; and
f) a pharmaceutically effective amount of an MCL1 inhibitor, or a pharmaceutically acceptable salt thereof.

In some embodiments of the method of treating venetoclax-resistant Acute Myeloid Leukemia in a human subject wherein a PTPN11 mutation is present, the MCL1 inhibitor administered to the human subject in need thereof is AZD5991, or a pharmaceutically acceptable salt thereof.

Within each of the methods above for diagnosing and treating venetoclax-resistant Acute Myeloid Leukemia in a human subject, there is another embodiment further comprising a step of detecting whether a high level of expression of CLEC7A (CD369) is present in the sample, wherein a high level of BCL2A1 expression is indicative of or supports a venetoclax-resistant diagnosis.

Within each of the methods above for diagnosing and treating venetoclax-resistant Acute Myeloid Leukemia in a human subject, there is another embodiment further comprising a step of detecting whether a high level of expression of BCL2A1 is present in the sample, wherein a high level of BCL2A1 expression is indicative of or supports a venetoclax-resistant diagnosis. In a human subject in which a high level of expression of BCL2A1 is present, a BCL2A1 inhibitor may be added to the Acute Myeloid Leukemia treatment regimen.

In addition, within each of the methods above for diagnosing and treating venetoclax-resistant Acute Myeloid Leukemia in a human subject, there is another embodiment further comprising a step of detecting whether a high level of expression of CD369 is present in the sample, wherein a high level of CD369 expression is indicative of or supports a venetoclax-resistant diagnosis. In a human subject in which a high level of expression of CD369 is present, a CD369 inhibitor may be added to the Acute Myeloid Leukemia treatment regimen.

Within each of the methods above for diagnosing and treating venetoclax-resistant Acute Myeloid Leukemia in a human subject, there is another embodiment further comprising a step of detecting whether a high level of expression of both of CLEC7A (CD369) and BCL2A1 is present in the sample, wherein a high level of BCL2A1 expression is indicative of or supports a venetoclax-resistant diagnosis.

Within each of the methods above for diagnosing and treating venetoclax-resistant Acute Myeloid Leukemia in a human subject, there is another embodiment further comprising a step of detecting whether a high level of expression of CD14 is present in the sample, wherein a high level of CD14 expression is indicative of or supports a venetoclax-resistant diagnosis.

Within each of the methods above for diagnosing and treating venetoclax-resistant Acute Myeloid Leukemia in a human subject, there is another embodiment further comprising a step of detecting whether a PML-RARA translocation, a WT1 mutation, or a FLT3 with IDH1 mutation is present in the biological sample, wherein absence of them further indicates or supports a venetoclax-resistant diagnosis.

Within each of the methods of treating venetoclax-resistant AML above, there is a further embodiment wherein the subject in need thereof is administered a pharmaceutically effective amount of venetoclax, or a pharmaceutically acceptable salt thereof, and a pharmaceutically effective amount of at least one therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

Within each of the methods of treating venetoclax-resistant AML above, there is a further embodiment wherein the subject in need thereof is administered a pharmaceutically effective amount of venetoclax, or a pharmaceutically acceptable salt thereof, and a pharmaceutically effective amount of at least two therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

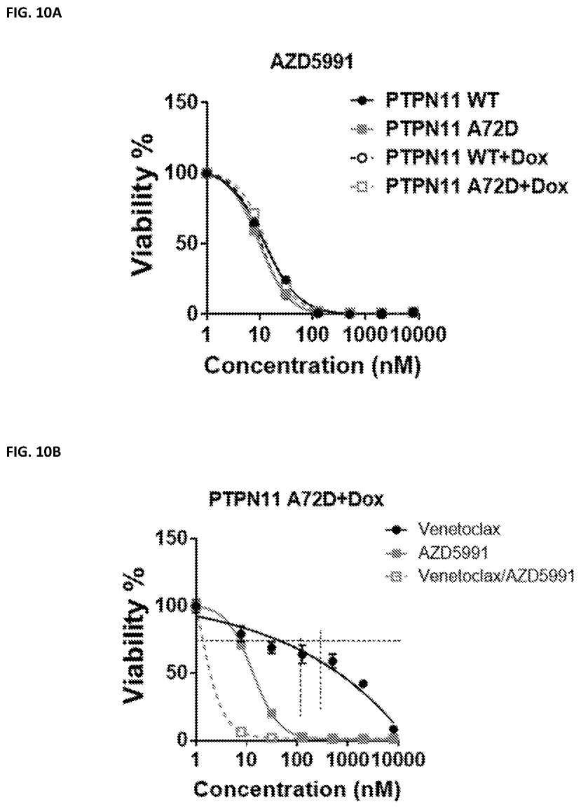

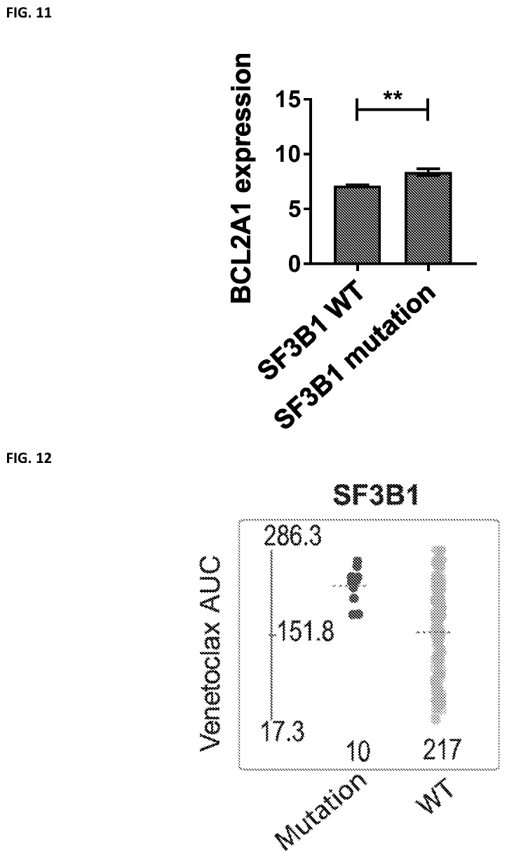

FIGS. 10(a) and 10(b) represent viability assays showing PTPN11 mutation is sensitive to AZD5991 and AZD5991 in combination with venetoclax. The graph of FIG. 10(a) depicts the cell viabilities of an inducible PTPN11 WT or A72D mutant transduced cells in the presence of AZD5991 (MCL-1 inhibitor) with or without doxycycline (Dox). The graph of FIG. 10(b) depicts the cell viabilities of the PTPN11 A72D mutant induced by Doxycycline, in the presence of AZD5991 (MCL-1 inhibitor) alone, venetoclax alone, or in combination.

### Venetoclax-Sensitive AML

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of one or more additional agents selected from the group of ibrutinib, sorafenib, dasatinib, doramapimod (BIRB 796), quizartinib, and trametinib, or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of ibrutinib, or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of sorafenib, or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of doramapimod (BIRB 796), or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of quizartinib, or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of dasatinib, or a pharmaceutically acceptable salt thereof.

Provided is a method of treating venetoclax-sensitive AML in a subject, the method comprising administering to the subject in need thereof a pharmaceutically effective amount of venetoclax, or a pharmaceutically acceptable salt thereof, and a pharmaceutically effective amount of trametinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of one or more additional agents selected from the group of ibrutinib, sorafenib, dasatinib, doramapimod (BIRB 796), quizartinib, and trametinib, or a pharmaceutically acceptable salt thereof.

Also provided is a method of diagnosing and treating venetoclax-sensitive Acute Myeloid Leukemia in a human subject, the method comprising:
e) obtaining a biological sample from the human subject;
f) detecting whether a PML-RARA translocation, a WT1 mutation, or a FLT3 with IDH1 mutation is present in the biological sample;
g) diagnosing the human subject with venetoclax-sensitive Acute Myeloid Leukemia when one or more of a PML-RARA translocation, a WT1 mutation, or a FLT3 with IDH1 mutation is detected in the biological sample; and
h) administering to the human subject in need thereof:
   iii) a pharmaceutically effective amount of venetoclax, or a pharmaceutically acceptable salt thereof; and
   iv) a pharmaceutically effective amount of trametinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of ibrutinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of sorafenib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of dasatinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of doramapimod (BIRB 796), or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a PML-RARA translocation, a WT1 mutation, or a FLT3 with IDH1 mutation is present in the biological sample;
c) diagnosing the human subject with venetoclax-sensitive Acute Myeloid Leukemia when one or more of a PML-RARA translocation, a WT1 mutation, or a FLT3 with IDH1 mutation is detected in the biological sample; and
d) administering to the human subject in need thereof:
e) a pharmaceutically effective amount of venetoclax, or a pharmaceutically acceptable salt thereof; and
f) a pharmaceutically effective amount of quizartinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of one or more additional agents selected from the group of ibrutinib, sorafenib, dasatinib, doramapimod (BIRB 796), quizartinib, and trametinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of trametinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of ibrutinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount sorafenib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of dasatinib, or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of doramapimod (BIRB 796), or a pharmaceutically acceptable salt thereof.

Also provided is a method of diagnosing and treating venetoclax-sensitive Acute Myeloid Leukemia in a human subject, the method comprising:
a) obtaining a biological sample from the human subject;
b) detecting whether a low expression level of one or more genes selected from CD369, CD14, and BCL2A1 is present in the biological sample;
c) diagnosing the human subject with venetoclax-sensitive Acute Myeloid Leukemia when a low expression level of one or more genes selected from CD369, CD14, and BCL2A1 is detected in the biological sample; and
d) administering to the human subject in need thereof:
e) a pharmaceutically effective amount of venetoclax, or a pharmaceutically acceptable salt thereof; and
f) a pharmaceutically effective amount of quizartinib, or a pharmaceutically acceptable salt thereof.

In each of the methods herein for diagnosing and treating a human subject, it is understood that the “human subject in need thereof” indicated to receive administration of one or more agents is the human diagnosed with the condition indicated to be treated. The term “human subject in need thereof” in those appearances may be used interchangeably with “human subject diagnosed with venetoclax-resistant Acute Myeloid Leukemia”, “human so diagnosed”, or other like description.

In another embodiment of the methods herein of diagnosing and treating venetoclax-sensitive or venetoclax-resistant Acute Myeloid Leukemia in a human subject provided above, there is another embodiment comprising the same steps, but further comprising detecting whether a high level of BCL2A1 expression or a low level of BCL2A1 expression is present in the biological sample from the human subject, such as a blood sample, wherein a low expression of BCL2A1 in the biological sample further indicates or supports a diagnosis of venetoclax sensitivity, and a high level of BCL2A1 expression in the biological sample further indicates or supports a diagnosis of venetoclax-resistance.

For gene expression, we observed that venetoclax correlated with 3 gene expression clusters (coefficient frequency: 0.94, 0.80 and 0.71 respectively) among 21 gene expression clusters in AML, associated with innate immune system, neutrophil degranulation, and interleukin-10 signaling. Among the BCL2 gene family, venetoclax AUC positively correlated with BCL2A1 (r=0.59, p<0.0001) and MCL1 (r=0.26, p=0.001) expression, whereas it negatively correlated with BCL2 (r=−0.53, p<0.0001) expression. BCL2A1 is the only BCL2 family gene within all three clusters and correlated the best with venetoclax sensitivity. Interestingly, within the three gene clusters, we observed that cell surface markers CLEC7A (CD369) and CD14 correlated with venetoclax sensitivity (r=0.68 and 0.64, p<0.0001). AML patient samples expressing CD14 detected by flow cytometry also demonstrated reduced venetoclax sensitivity (p=0.005), which could potentially serve as a biomarker to identify venetoclax resistant patients. For cytogenetic categories and somatic mutations, we observed that AML samples harboring PML-RARA translocations, WT1, and FLT3 with IDH1 mutations are more sensitive to venetoclax, and samples with TET2, KRAS, PTPN11 and SF3B1 mutations are more resistant. We validated the effect of WT1, KRAS, and PTPN11 mutations on venetoclax sensitivity by performing drug assays on mouse bone marrow stem cells and/or AML cell lines overexpressing each mutant. Samples harboring PTPN11 mutations demonstrated high MCL-1 expression, and PTPN11 mutant-transduced cells remain sensitive to Idasanutlin, which was previously shown to downregulate MCL-1 expression. Samples with KRAS mutations demonstrated high BCL2A1 expression, which potentially mediate venetoclax resistance. For venetoclax drug combinations, we observed that venetoclax-trametinib demonstrated a synergistic effect on samples that are sensitive to venetoclax, whereas venetoclax-palbociclib, venetoclax-Array-382, venetoclax-sorafenib, venetoclax-ruxolitinib, venetoclax-dasatinib, and venetoclax-idelalisib are active against samples that are resistant to venetoclax, indicating potential therapeutic combinations. Interestingly, the CDK inhibitor palbociclib demonstrated no effect on the majority of AML samples and does not correlate with the BCL2A1 expression as a single agent, yet shows the most robust synergy with venetoclax, especially on samples that are resistant to venetoclax and with high BCL2A1 expression, indicating a potential synthetic lethal interaction. Venetoclax-palbociclib AUC also negatively correlated with CLEC7A and CD14 expression, indicating that venetoclax-palbociclib could circumvent venetoclax resistance to treat patients with high CLEC7A and/or CD14 expression.

In summary, we have identified that CD14 and/or CLEC7A could be used as biomarkers to predict venetoclax sensitivity in AML, and we propose to combine venetoclax and palbociclib to treat patients with a venetoclax resistant profile (high CD14/CLEC7A expression or high BCL2A1 expression, or presence of KRAS mutations).

The table below summarizes the coefficient frequencies of gene expression clusters and mutations associated with venetoclax drug area under the curve (AUC) measured by lasso approach (Tibshirani, 1996). The higher the frequency, the better the correlation.

| Datetype | Coefficient | Frequency |
|---|---|---|
| Expression | brown | 0.943 |
| Expression | greenyellow | 0.798 |
| Expression | turquoise | 0.713 |
| Mutation | TET2 | 0.540 |
| Expression | light green | 0.462 |
| Mutation | KRAS | 0.302 |
| Expression | lightyellow | 0.284 |
| Expression | red | 0.218 |
| Mutation | ASXL1 | 0.212 |
| Mutation | NPM1 | 0.205 |
| Mutation | WT1 | 0.192 |
| Mutation | RUNX1 | 0.190 |
| Expression | royalblue | 0.186 |
| Expression | green | 0.186 |
| Mutation | IDH2 | 0.158 |
| Expression | cyan | 0.142 |
| Mutation | IDH1 | 0.138 |
| Mutation | CEBPA | 0.124 |
| Mutation | NRAS | 0.118 |
| Mutation | DNMT3A | 0.116 |
| Mutation | FLT3 | 0.108 |
| Mutation | FLT3_ITD | 0.103 |
| Expression | grey60 | 0.099 |
| Mutation | SRSF2 | 0.094 |
| Expression | blue | 0.90 |
| Expression | salmon | 0.077 |
| Mutation | TP53 | 0.076 |
| Expression | black | 0.073 |
| Expression | yellow | 0.071 |
| Expression | pink | 0.070 |
| Expression | lightcyan | 0.069 |

The table below provides reactome pathway analysis of genes from brown, greenyellow and turquoise clusters.

| Pathway Name | pValue | FDR |
|---|---|---|
| Neutrophil degranulation | 1.11E−16 | 4.6E−14 |
| Innate Immune System | 4.00E−15 | 8.2E−13 |
| Immune System | 1.12E−10 | 1.5E−08 |
| Interleukin-10 signaling | 1.81E−04 | 1.8E−02 |
| Immunoregulatory interactions between | 5.74E−05 | 1.6E−02 |
| a lymphoid and a Non-lymphoid cell | | |
| Diseases of Immune System | 3.01E−04 | 4.1E−02 |
| Diseases associated with TLR signaling cascade | 3.01E−04 | 4.1E−02 |

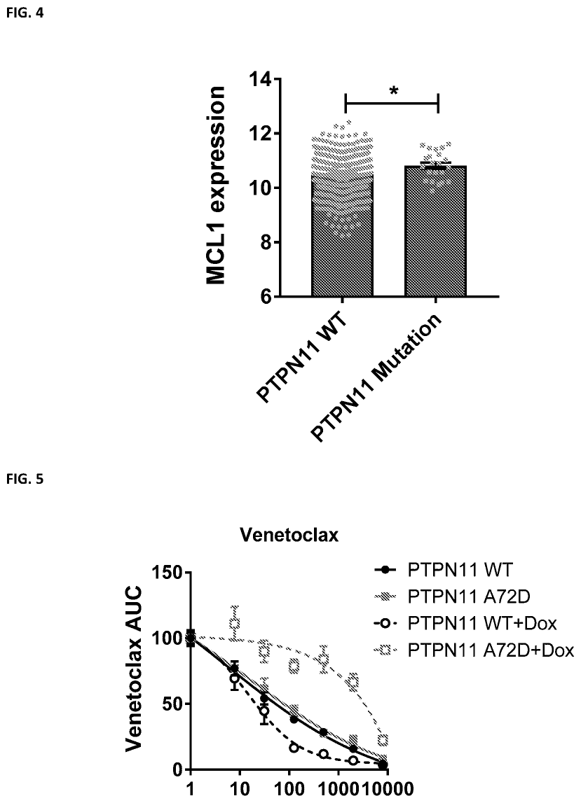

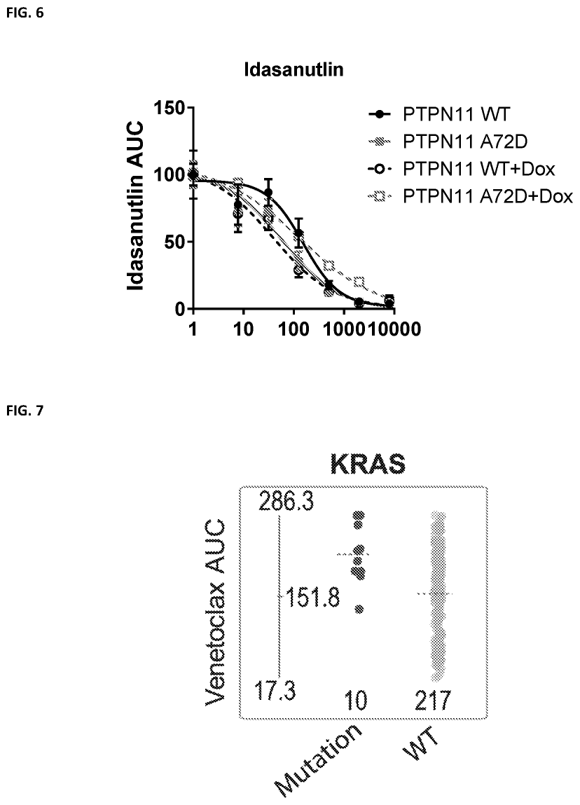

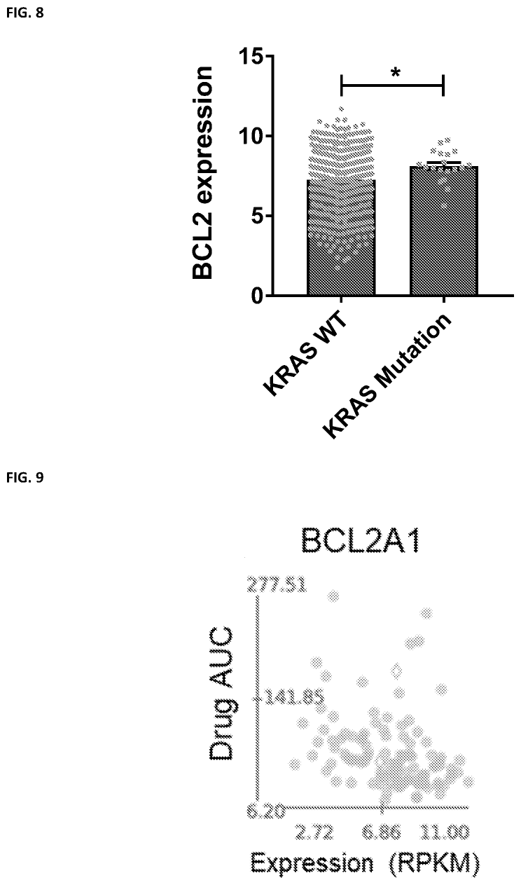

The graph of FIG. 3 demonstrates higher venetoclax AUCs in AML samples with PTPN11 mutations compared to samples with wild type (WT) PTPN11. Graphs depict the mean±SEM of cell viabilities of a inducible PTPN11 WT or A72D mutant transduced cells in the presence of venetoclax (FIG. 4), or idasanutlin (FIG. 5), with or without doxycycline (Dox) for 72 hours detected by MTS assay.

### Samples

Mononuclear cells were isolated by Ficoll gradient centrifugation from freshly obtained bone marrow aspirates or peripheral blood draws. Cell pellets were snap frozen in liquid nitrogen for subsequent DNA isolation (Qiagen, DNeasy Blood & Tissue Kit), freshly pelleted cells were lysed immediately in GTC lysate for subsequent RNA isolation (Qiagen, RNeasy Mini Kit), and freshly isolated mononuclear cells were plated into an ex vivo drug sensitivity assays within 24 hours (described in detail below). Genetic characterization of the leukemia samples included results of a clinical deep-sequencing panel of genes commonly mutated in hematologic malignancies (Sequenome and GeneTrails (OHSU); Foundation Medicine (UTSW); Genoptix; and Illumina).

### Ex Vivo Functional Drug Screens

Ex vivo functional drug screens were performed on freshly isolated mononuclear cells from AML samples as previously described (Kurtz et al., 2017; Tyner et al., 2018). Briefly, 10,000 cells per well were arrayed into three, 384-well plates containing venetoclax and venetoclax combinations. Drug plates were created using inhibitors purchased from LC Laboratories and Selleck Chemicals and master stocks were reconstituted in DMSO and stored at −80° C. Inhibitors were distributed into 384-well plates prepared with a single agent per well in a seven-point concentration series ranging from 10 μM to 0.0137 μM for each drug (except dasatinib, which was plated at a concentration range of 1 μM to 0.00137 μM). Similar plates were prepared with indicated pairwise inhibitor combinations in seven-point fixed molar concentration series identical to those used for single agents. DMSO control wells and positive control wells containing a drug combination of Flavopiridol, Staurosporine and Velcade were placed on each plate, with the final concentration of DMSO ≤0.1% in all wells. PBMC were plated across single-agent inhibitor panels within 24 h of collection. Cells were seeded into 384-well assay plates at 10,000 cells per well in RPMI 1640 media supplemented with FBS (10%), I-glutamine, penicillin/streptomycin, and β-mercaptoethanol (10⁻⁴ M). After 3 d of culture at 37° C. in 5% CO2, MTS reagent (CellTiter96 AQueous One; Promega) was added, optical density was measured at 490 nm, and raw absorbance values were adjusted to a reference blank value and then used to determine cell viability (normalized to untreated control wells). For cell line drug screening, 1000 cells per well were seeded. A nine-point concentration series ranging from 6 μM to 0.9 nM for each drug were used.

### Drug IC50 and Drug Area Under the Curve (AUC)

A ‘curve-free’ AUC (integration based on fine linear interpolation between the 7 data points themselves) was calculated for those runs with within-panel replicates after applying a ceiling of 100 and a floor of 0 for the normalized viability.

Based on the methodology used in our prior drug combination study (Kurtz et al., 2017; Tyner et al., 2018), a probit regression was fit to all possible run groups using the model:

(normalized_viability/100)˜1+log 10(concentration)

Where for all groups there were N=7 dose-response measurements.

The summary measures of curve fit were inspected and cutoffs were devised removing all runs with an AIC>12 and deviance>2. Finally, these data were compared to the AUC values from third order polynomial fits. Those runs that were discrepant in terms of sensitive/resistant calls were manually reviewed as subject to removal. The IC₅₀ was defined as the lowest concentration to achieve 50% predicted viability and the AUC was computed by integration of the curve height across the tested dose range.

### Retroviral Vector Production and Transduction

PTPN11 mutation was generated using the QuikChange II XL site-directed mutagenesis kit (Agilent Technologies) on a pENTR vector. The wide type (WT) and mutant pENTR vectors were cloned into a gateway compatible Tet-inducible lentiviral vector, pinducer20 (Addgene, #444012) via Gateway Cloning System (Invitrogen). Lentivirus was produced by transfecting HEK 293T/17 cells together with psPAX2 (psPAX2 was a gift from Didier Trono (Addgene plasmid #12260) and pLP/VSVG (Invitrogen). After 2 days, the virus containing supernatants were filtered, and infected to cells followed by flow cytometry (FACS) sorting.

### Exome Sequencing and RNA Sequencing

Exome sequencing was performed on a HiSeq 2500 using Illumina Nextera capture probes and paired end 100 cycle protocols as previously described (Tyner et al., 2018). For RNA-Seq, libraries were constructed using the SureSelect stranded RNA-seq protocol (Agilent) on the Bravo robot (Agilent) and sequenced on the HiSeq 2500 using a 100 cycle paired end protocol as previously described (Tyner et al., 2018).

### Statistical Analysis

Statistical analyses were performed on GraphPad Prism software. The data were expressed as the mean±standard error of mean (MED). Statistical significance was determined using Student's two-tailed t tests and expressed as p-values (*p<0.05 and **p<0.01).

### REFERENCES

Kurtz, S. E., Eide, C. A., Kaempf, A., Khanna, V., Savage, S. L., Rofelty, A., English, I., Ho, H., Pandya, R., Bolosky, W. J., et al. (2017). Molecularly targeted drug combinations demonstrate selective effectiveness for myeloid- and lymphoid-derived hematologic malignancies. Proc. Natl. Acad. Sci. U. S. A. 114, E7554-E7563.
Tyner, J. W., Yang, W. F., Bankhead, A., Fan, G., Fletcher, L. B., Bryant, J., Glover, J. M., Chang, B. H., Spurgeon, S. E., Fleming, W. H., et al. (2013). Kinase pathway dependence in primary human leukemias determined by rapid inhibitor screening. Cancer Res. 73, 285-296.
Tyner, J. W., Tognon, C. E., Bottomly, D., Wilmot, B., Kurtz, S. E., Savage, S. L., Long, N., Schultz, A. R., Traer, E., Abel, M., et al. (2018). Functional genomic landscape of acute myeloid leukaemia. Nature 562, 526-531.

### Therapeutic Agents

Venetoclax is marketed by Abbvie/Genentech under the tradename VENCLEXTA® as oral tablets in 10 mg, 50 mg, and 100 mg dosages. Venetoclax may be administered in once or twice daily doses in the methods and regimens herein at from about 10 mg once or twice per day to about 500 mg once or twice daily. In some embodiments, venetoclax administration is initiated at from about 10 mg per day to about 20 mg once or twice per day and increased weekly to a final desired dosage. In some embodiments, venetoclax is administered to a subject at from about 10 mg once or twice per day to about 20 mg once or twice per day during the first week of administration, about 50 mg once or twice per day in the second week, about 100 mg once or twice per day during the third week, about 200 mg once or twice per day during the fourth week, and 400 mg once or twice per day for the fifth and subsequent weeks. In other embodiments, venetoclax may be administered at a dose of 100 mg on day 1, 200 mg on day 2, 400 mg on day 3, and 600 mg on day 4 and 600 mg per day thereafter. In other embodiments, venetoclax may be administered as just described for days 1-4, except that after the 600 mg on day 4, the day 5 dose is raised to 800 mg and the dose is maintained at 800 mg per day thereafter. In other escalating regimens, the doses will be raised directly from the 400 mg day 3 dose to 800 mg on day 4 and maintained at 800 mg daily thereafter.

Oncology agent palbociclib is marketed by Pfizer Inc. under the tradename IBRANCE™ in 75 mg, 100 mg, and 125 mg capsule form. In the methods herein, palbociclib may be administered in doses of from about 75 mg to about 150 mg once or twice daily. In some embodiments, the palbociclib is administered to the subject in need thereof at a daily dose of from about 50 mg to about 300 mg. In other embodiments, palbociclib is administered at a dose of from about 75 mg to about 150 mg per day. In separate embodiments, palbociclib is administered at a daily dose of 50 mg, 75 mg, 100 mg, 125 mg, and 150 mg. In some dosing regimens, palbociclib may be administered to human subjects orally at such doses for a 21-day period, followed by a 7-day period off-treatment. In other dosing regimens, palbociclib may be administered to human subjects orally at such doses for a 28-day period, followed by a 7-day period off-treatment.

ARRY-382 (Array 382, CAS Registry No. 1361232-61-2) is a highly selective oral small-molecule inhibitor of colony-stimulating factor-1 receptor (CSF1R) and may be administered in once or twice daily doses in the methods and regimens herein at from about 5 mg once or twice per day to about 500 mg once or twice per day. In some embodiments, the dosing in human subjects is from about 10 mg once or twice per day to about 400 mg once or twice per day. In other embodiments, the dosage administered once or twice per day is from about 25 mg to about 250 mg. In other embodiments, the dosage is given at from about 25 mg to about 200 mg once or twice daily. In some embodiments, the dosage is from about 250 mg daily to about 350 mg daily. In separate embodiments, the daily dosage of ARRY-382 in the methods herein is 25 mg, 50 mg, 100 mg, 150 mg, 200 mg, 250 mg, 300 mg, 350 mg, 400 mg, 450 mg, and 500 mg.

Sorafenib tosylate tablets are marketed under the NEXAVAR® tradename by Bayer HealthCare Pharmaceuticals, Inc. in 200 mg tablets. Sorafenib may be administered to human subjects at dosages of from about 200 mg once or twice daily to about 400 mg twice daily without food. In separate embodiments, sorafenib may be administered in the methods and regimens herein at a dose of 400 mg once per day, 400 mg twice per day, 400 mg once every other day, 200 mg per day, 200 mg twice per day, and 200 mg every other day.

Kinase inhibitor ruxolitinib (INCB018424) is marketed under the JAKAFI® tradename by Incyte as 5 mg, 10 mg, 15 mg, 20 mg, and 25 mg oral tablets. Administration of ruxolitinib may be from about 5 mg taken once daily to about 50 mg taken twice daily. In some embodiments, ruxolitinib will be administered to a subject in need thereof at a dose of from about 20 mg twice per day to about 30 mg twice per day. In some embodiments, ruxolitinib will be administered to the subject at 25 mg twice per day. In some embodiments, ruxolitinib will be administered to the subject at 50 mg twice per day. In separate embodiments, ruxolitinib will be administered to the subject at doses of 5 mg, 10 mg, 15 mg, 20 mg, and 25 mg, once daily for each. In separate embodiments, ruxolitinib will be administered to the subject at doses of 5 mg, 10 mg, 15 mg, 20 mg, and 25 mg, twice daily for each.

The dual BCR/ABL and Src family tyrosine kinase inhibitor dasatinib is marketed under the SPRYCEL® tradename by Bristol-Meyers Squibb and Otsuka America Pharmaceutical, Inc. in 20 mg, 50 mg, 70 mg, 80 mg, 100 mg, and 140 mg oral tablets. Administration of dasatinib in the methods and regimens herein may be from about 10 mg once or twice per day to about 150 mg once or twice per day. In one embodiment, dasatinib is administered to a subject at from about 25 mg per day to about 100 mg per day and venetoclax is administered to the subject at a dosage of from about 50 mg per day to about 400 mg per day. In another embodiment, dasatinib is administered to a subject at from about 25 mg per day to about 75 mg per day and venetoclax is administered to the subject at a dosage of from about 50 mg per day to about 300 mg per day. Another embodiment provides daily doses of 50 mg of dasatinib to the subject in combination with 200 mg daily doses of venetoclax.

Gilead Sciences, Inc. markets idelalisib (GS-1101, CAL-101) under the ZYDELIG® tradename in 100 mg and 150 mg tablets. In the present methods and dosing regiments, idelalisib may be administered to a subject in need thereof at doses of from about 25 mg to about 200 mg once or twice daily. In some embodiments, idelalisib may be administered in 50 mg or 100 mg doses once or twice daily. In other embodiments, idelalisib may be administered in 150 mg doses once or twice daily. In some embodiments, idelalisib is administered to the subject at a dose of 150 mg twice daily.

GlaxoSmithKline markets trametinib under the MEKINIST® tradename in 0.5 mg, 1 mg, and 2 mg oral tablets. Trametinib may be administered in the present methods once daily at a dosage of from about 0.5 mg to about 2.5 mg. In some embodiments, trametinib is administered to the subject in need thereof at a dose of 2 mg once per day.

Doramapimod (BIRB 796), or a pharmaceutically acceptable salt thereof, may be administered in the methods herein at a dose of from about 10 mg to about 200 mg once or twice daily. In some embodiments, doramapimod is administered at a dose of from about 40 mg to about 150 mg once or twice daily.

Quizartinib, or a pharmaceutically acceptable salt thereof, may be administered in the methods herein at a dose of from about 5 mg to about 200 mg once or twice daily. In some embodiments, quizartinib is administered at a dose of from about 10 mg to about 80 mg once or twice daily.

AZD5991 is identified as the compound of Chemical Abstracts Registry No. 2143061-81-6, which may be called (Z)-16-chloro-11,21,25,61-tetramethyl-11H,21H,61H-10-oxa-4,8-dithia-1(7,3)-indola-2(4,3),6(3,5)-dipyrazola-9(3,1)-naphthalenacyclotridecaphane-12-carboxylic acid. In some embodiments, AZD5991 may be administered to a subject in need thereof at a dose of from about 1 mg/kg to about 200 mg/kg. In other embodiments, AZD5991 may be administered at a dose of from about 30 mg/kg to about 150 mg/kg. In other embodiments, AZD5991 may be administered at a dose of from about 50 mg/kg to about 120 mg/kg.

### Definitions

In one specific, non-limiting example, “high expression” or a “high level of expression” of a biomarker, for example a cell surface marker (such as, but not limited to, CD369 (also known as CLEC7A) or CD14) or an intracellular biomarker (such as, but not limited to, BCL2A1) can be described in terms of RNA-sequencing (RNA-seq) RPKM (reads per kilobase of transcript, per million mapped reads) values. For example, high biomarker expression can be described as a value equal to or greater than the median RPKM for that particular biomarker, whereas low biomarker expression can be described as a value below the median RPKM for that particular biomarker.

In another specific, non-limiting example, high expression or a high level of a biomarker on a resistant cell can be described in terms of an RPKM value for each transcript that falls within the 20% of samples with the highest area under the curve (AUC) for a particular drug. In yet another specific, non-limiting example, high expression of a biomarker on a sensitive cell can be described in terms of an RPKM value for each transcript that falls within the 20% of samples with the lowest AUC for a particular drug.

The modifier “about” used in connection with a quantity is inclusive of the stated value and has the meaning dictated by the context (e.g., includes the degree of error associated with measurement of the particular quantity). In some embodiments, the term refers to the stated value±10%. In other embodiments, it refers to the stated value±5%.

The terms “therapeutically effective amount” or “pharmaceutically effective amount” can be used interchangeably and refer to an amount of an agent that is sufficient to effect treatment, as defined below, when administered to a subject (e.g., a mammal, such as a human subject) in need of such treatment. The therapeutically or pharmaceutically effective amount will vary depending upon the subject and disease condition being treated, the weight and age of the subject, the severity of the disease condition, the manner of administration and the like, which can readily be determined by one of ordinary skill in the art. For example, a “therapeutically effective amount” or a “pharmaceutically effective amount” of a compound described herein, or a pharmaceutically acceptable salt or co-crystal thereof, is an amount sufficient to reduce AML cells or their activity, and thereby treat a subject (e.g., a human subject) suffering AML, or to ameliorate or alleviate the existing symptoms of AML. For example, a therapeutically or pharmaceutically effective amount may be an amount sufficient to decrease a symptom of AML.

“Treatment” or “treating” is an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival). Particularly included herein is the treatment comprising inhibiting, slowing or arresting, preventing or delaying the spread of, relieving, or providing total or partial remission of venetoclax-resistant AML or enhancing the effect of another therapeutic agent to do so.

“Delaying” the development of a disease or condition means to defer, hinder, slow, retard, stabilize, and/or postpone development of venetoclax-resistant AML. This delay can be of varying lengths of time, depending on the history of the condition, and/or subject being treated. A method that “delays” development of the condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, blood tests, and/or bone marrow aspiration or biopsy. Development may also refer to disease or condition progression that may be initially undetectable and includes occurrence, recurrence, and onset.

The term “venetoclax-resistance” refers to an aspect of a disease or condition in a subject, such as an acute myeloid leukemia, wherein it is resistant to the pharmacological activity of initial venetoclax treatment (primary resistance) or there is a reduction in effectiveness over time resulting from the treatment (acquired or evolved resistance), such that the pharmacologic activity of venetoclax is insufficient to successfully treat the disease or condition. The term “venetoclax-sensitive” refers to a disease or condition in a subject, such as an acute myeloid leukemia, that may be successfully treated by venetoclax administration, i.e. in the case of AML, cancer cells are responsive to one or more administrations of venetoclax.

“Pharmaceutically acceptable salts” include, for example, salts with inorganic acids and salts with an organic acid. Examples of salts may include hydrochloride, phosphate, diphosphate, hydrobromide, sulfate, sulfinate, nitrate, malate, maleate, fumarate, tartrate, succinate, citrate, acetate, lactate, methanesulfonate (mesylate), benzenesuflonate (besylate), p-toluenesulfonate (tosylate), 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

“Subject” refers to an animal, such as a mammal, that has been or will be the object of treatment, observation or experiment. The methods described herein may be useful in both human therapy and veterinary applications. In some embodiments, the subject is a mammal; in some embodiments the subject is human; and in some embodiments the subject is chosen from cats and dogs. “Subject in need thereof”, “human subject in need thereof”, or “human in need thereof” refers to a subject, such as a human, who may have or is suspected to have diseases or conditions that would benefit from certain treatment; for example treatment with a compound of Formula I, or a pharmaceutically acceptable salt or co-crystal thereof, as described herein. This includes a subject who may be determined to be at risk of or susceptible to such diseases or conditions, such that treatment would prevent the disease or condition from developing.

The term “stereoisomers” refers to compounds which have identical chemical constitution, but differ with regard to the arrangement of the atoms or groups in space.

The term “co-crystal” or “co-crystal salt” as used herein means a crystalline material composed of two or more unique solids at room temperature, each of which has distinctive physical characteristics such as structure, melting point, and heats of fusion, hygroscopicity, solubility, and stability. A co-crystal or a co-crystal salt can be produced according to a per se known co-crystallization method. The terms co-crystal (or cocrystal) or co-crystal salt also refer to a multicomponent system in which there exists a host API (active pharmaceutical ingredient) molecule or molecules, such as a compound of Formula I, and a guest (or co-former) molecule or molecules. In particular embodiments the pharmaceutically acceptable co-crystal of the compound of Formula I or of the compound of Formula II with a co-former molecule is in a crystalline form selected from a malonic acid co-crystal, a succinic acid co-crystal, a decanoic acid co-crystal, a salicylic acid co-crystal, a vanillic acid co-crystal, a maltol co-crystal, or a glycolic acid co-crystal. Co-crystals may have improved properties as compared to the parent form (i.e., the free molecule, zwitter ion, etc.) or a salt of the parent compound. Improved properties can include increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like.

“BCL2A1” is the gene that encodes in humans for the Bcl-2-related protein A1. “Bcl-w” pr “BCL-w” refers to Bcl-2-like protein 2, which is a protein coded for by the BCL2L2 gene. Inhibitors of BCL-w include Navitoclax (ABT-263) and ABT-737.

“CLEC7A” refers to C-type lectin domain family 7 member a or Dectin-1 protein coded in humans by the CLEC7A gene.

“FAB” refers to the French-American-British classification of AML, including FAB subtypes M3 (acute promyelocytic leukemia or APL), M4 (acute myelomonocytic leukemia), and M5 (acute monocytic leukemia).

“PML-RARA” is an acronym for promyelocytic leukemia/retinoici acid receptor alpha, an abnormal gene sequence rearrangement of genetic material from two separate chromosomes (chromosomal translocation).

A “KRAS G12D” mutation refers to a mutation in the KRAS gene resulting in a replacement of the glycine (G) amino acid at position 12 with an aspartic acid group (D). In some embodiments herein, the mutation in the KRAS gene to be detected is a KRAS G12D mutation.

A “PTPN11 A72D” mutation refers to a mutation in the protein tyrosine phosphatase, non-receptor type 11 (PTPN11) resulting in a replacement of the alanine (A) amino acid at position 72 with an aspartic acid group (D). In some embodiments herein, the mutation in the PTPN11 gene to be detected is a PTPN11 A72D mutation.

The term “Beat AML” refers to a collaboration between The Leukemia and Lymphoma Society and the OHSU Knight Cancer Institute bringing together scientists from multiple disciplines to understand Acute Myeloid Leukemia. The “Beat AML cohort” refers to the cohort of patient specimens accrued for the collaboration.

Compositions (including, for example, formulations and unit dosages) comprising the therapeutic agents described herein, or a pharmaceutically acceptable salt or co-crystal thereof, can be prepared and placed in an appropriate container, and labeled for treatment of an indicated condition. Accordingly, provided is also an article of manufacture, such as a container comprising one or more unit dosage forms of venetoclax, or a pharmaceutically acceptable salt or co-crystal thereof, one or more unit dosage forms of one or more of the therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt or co-crystal thereof,and a label containing instructions for use of the therapeutic agents in the treatment of Venetoclax-resistant AML.

In some embodiments, the article of manufacture is a container comprising at least one unit dosage form of the pharmaceutical agent venetoclax and one or more agents selected from the group of palbociclib, ARRY-382 (Array 382), sorafenib, ruxolitinib, dasatinib, doramapimod (BIRB 796), quizartinib, and idelalisib, or a pharmaceutically acceptable salt or co-crystal thereof, and at least one pharmaceutically acceptable vehicle per each agent. The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container indicating directions for use in the treatment of AML. It should be understood that the active ingredient may be packaged in any material capable of improving chemical and physical stability, such as an aluminum foil bag. In some embodiments, diseases or conditions indicated on the label can include, for example, treatment of venetoclax-resistant AML.

Any pharmaceutical composition provided in the present disclosure may be used in the articles of manufacture, the same as if each and every composition were specifically and individually listed for use in an article of manufacture.

Also provided is a kit that includes a pharmaceutically effective amount of the therapeutic agent venetoclax, or a pharmaceutically acceptable salt, pharmaceutically acceptable co-crystal, pharmaceutically acceptable ester, stereoisomer, mixture of stereoisomers or tautomer thereof, and a pharmaceutically effective amount of one or more therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt, pharmaceutically acceptable co-crystal, pharmaceutically acceptable ester, stereoisomer, mixture of stereoisomers or tautomer thereof. In a further aspect, the kit may comprise a label and/or instructions for use of the therapeutic agents for the treatment of AML in a subject (e.g., human) in need thereof. In some embodiments, the disease or condition may be venetoclax-resistant AML.

Also provided is a kit that includes a pharmaceutically effective amount of the therapeutic agent venetoclax, or a pharmaceutically acceptable salt, pharmaceutically acceptable co-crystal, pharmaceutically acceptable ester, stereoisomer, mixture of stereoisomers or tautomer thereof, and a pharmaceutically effective amount of trametinib, or a pharmaceutically acceptable salt, pharmaceutically acceptable co-crystal, pharmaceutically acceptable ester, stereoisomer, mixture of stereoisomers or tautomer thereof. In a further aspect, the kit may comprise a label and/or instructions for use of the therapeutic agents for the treatment of AML in a subject (e.g., human) in need thereof. In some embodiments, the disease or condition may be venetoclax-sensitive AML.

Also provided are pharmaceutical compositions for use in the treatment of venetoclax-resistant AML, the compositions comprising a pharmaceutically effective amount of venetoclax, or a pharmaceutically acceptable salt thereof, pharmaceutically acceptable co-crystal, pharmaceutically acceptable ester, stereoisomer, mixture of stereoisomers or tautomer thereof, and a pharmaceutically effective amount of one or more therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt, pharmaceutically acceptable co-crystal, pharmaceutically acceptable ester, stereoisomer, mixture of stereoisomers or tautomer thereof.

Also provided is the use of venetoclax, or a pharmaceutically acceptable salt thereof, pharmaceutically acceptable co-crystal, pharmaceutically acceptable ester, stereoisomer, mixture of stereoisomers or tautomer thereof, and one or more therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt, pharmaceutically acceptable co-crystal, pharmaceutically acceptable ester, stereoisomer, mixture of stereoisomers or tautomer thereof, in the preparation of a medicament for use in the treatment of venetoclax-resistant AML.

### Result

### Factors Influencing Venetoclax Sensitivity

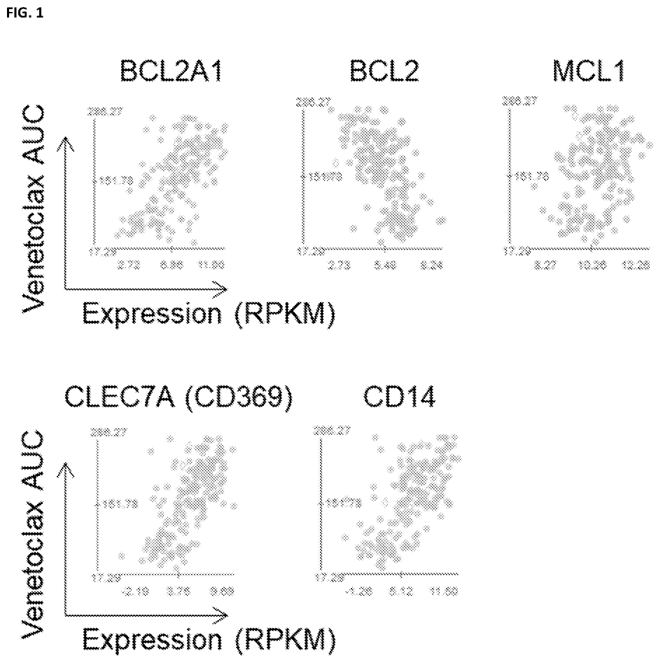

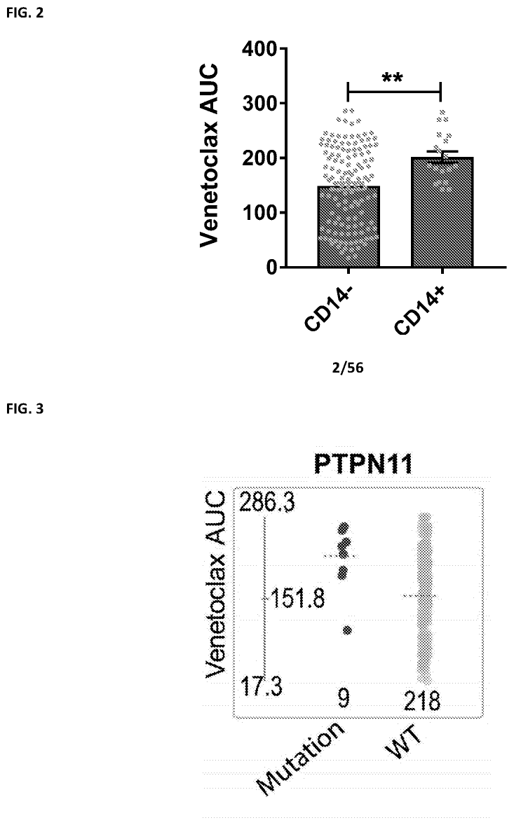

To determine whether sensitivity to venetoclax correlated with prominent clinical characteristics, gene expression, or genetic abnormalities found in AML patients, we analyzed patient samples from the Beat AML cohort (n=295) that had been subjected to venetoclax screening, many of which had whole-exome sequencing (WES), RNAseq, and detailed clinical annotations (FIG. 1A). We first compared the distribution of venetoclax sensitivity represented by area under the curve (AUC) to clinical characteristics and common chromosome translocations (FIGS. 1B-C and the table below).

Correlation between clinical parameters and venetoclax AUC

| | Person | P | P (Multi-test |
|---|---|---|---|
| Category | r | (two-tailed) | Corrected) |
| % Monocytes in PB | 0.436 | 6.89E−09 | 1.45E−07 |
| % Blasts in BM | −0.406 | 3.70E−07 | 7.78E−06 |
| % Blasts in PB | −0.351 | 7.42E−06 | 1.56E−04 |
| % Neutrophils in PB | 0.346 | 1.32E−05 | 2.78E−04 |
| % Eosinophils in PB | 0.221 | 7.39E−03 | 1.55E−01 |
| MCV | 0.207 | 2.08E−02 | 4.37E−01 |
| % Immature Granulocytes in PB | 0.203 | 5.02E−02 | 1.05E+00 |
| Platelet Count | 0.134 | 7.44E−02 | 1.56E+00 |
| Hematocrit | 0.137 | 8.86E−02 | 1.86E+00 |
| % Nucleated RBCs in PB | −0.168 | 1.03E−01 | 2.16E+00 |
| Hemoglobin | 0.124 | 1.24E−01 | 2.60E+00 |
| age at Diagnosis | 0.068 | 3.07E−01 | 6.45E+00 |
| Creatinine | 0.080 | 3.39E−01 | 7.11E+00 |
| % Basophils in PB | 0.078 | 3.71E−01 | 7.79E+00 |
| WBC Count | −0.062 | 3.94E−01 | 8.27E+00 |
| % Lymphocytes in PB | 0.063 | 4.42E−01 | 9.28E+00 |

Overall, we found a significant association between venetoclax resistance and low BM/peripheral blood (PB) blast count, high monocyte/neutrophil count, transformed AML, and FAB M4 and M5 AML subset. In contrast, AML with high blast count, FAB M3, and AML with PML-RARA translocations were more sensitive to venetoclax.

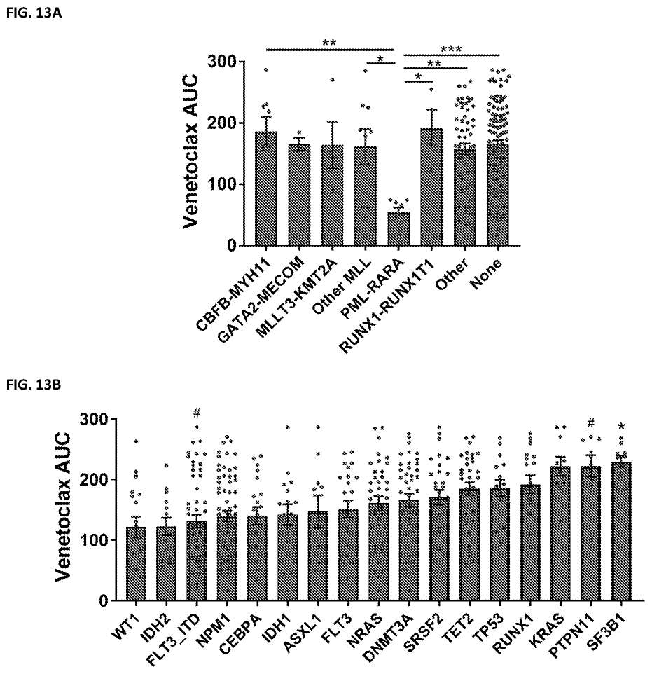

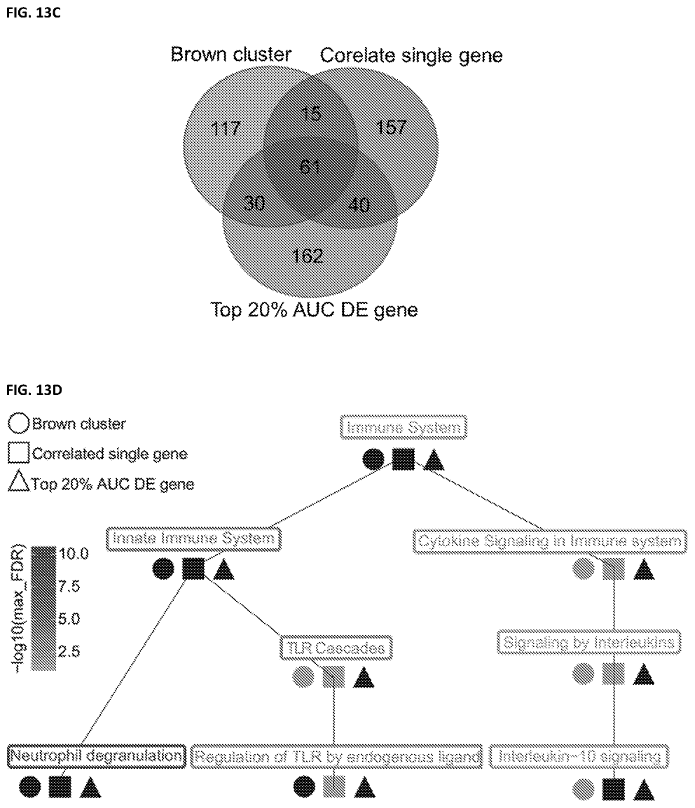

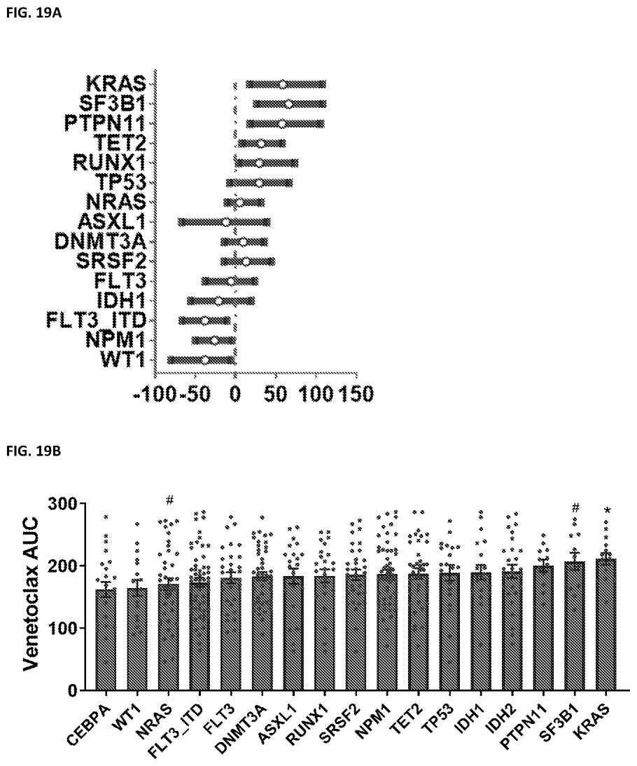

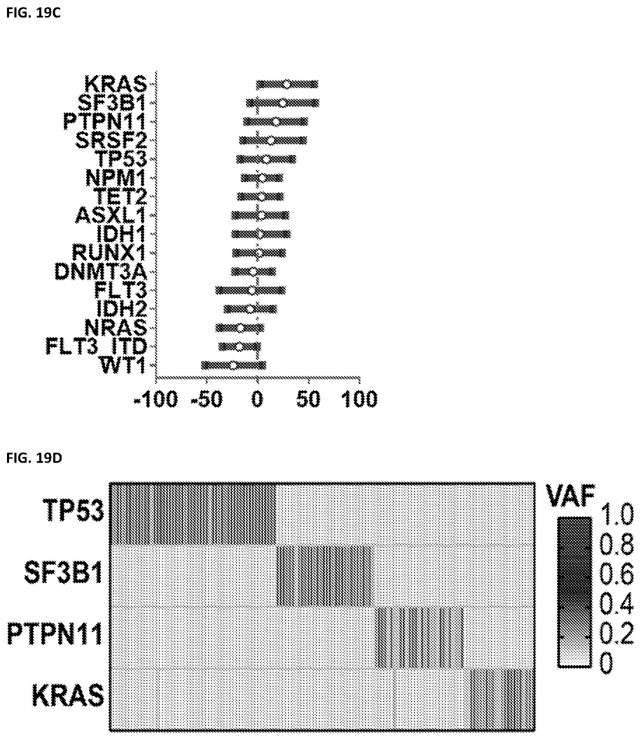

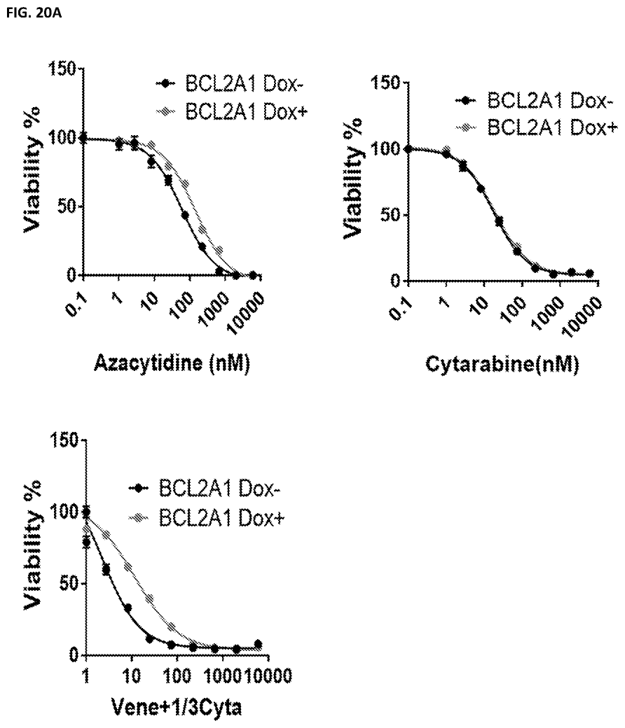

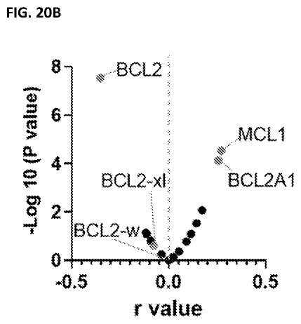

Next, we evaluated the relationship between venetoclax sensitivity and common AML mutations. We observed that cases with KRAS, PTPN11, and SF3B1 mutations were relatively resistant to venetoclax (as shown by higher AUCs and hazard ratios>1) (FIGS. 13B and 19A). Accordingly, these mutations also demonstrated decreased sensitivity to 4-{4-[(4′-Chloro-2-biphenylyl)methyl]-1-piperazinyl}-N-[(4-{[(2R)-4-(dimethylamino)-1-(phenylsulfanyl)-2-butanyl]amino}-3-nitrophenyl)sulfonyl]benzamide (ABT-737, a BCL2, BCL-w, and BCL-xL inhibitor) (FIGS. 19B-C). Notably, KRAS, PTPN11, and SF3B1 mutations are mutually exclusive in the Beat AML cohort (FIG. 19D). For gene expression, we identified 273 genes correlated with venetoclax AUC with r≥0.5 or r<−0.5 and FDR≤2E-10. We also correlated venetoclax sensitivity with the 14 gene clusters identified from the Beat AML cohort WGCNA analysis²⁹ (Supplementary Data 3). We observed that the “brown cluster” correlated best with drug sensitivity. Moreover, we compared gene expression between the most sensitive (the lowest 0-20^{th} percentile of AUC) and the most resistant (the highest 80^{th}-100^{th} percentile of AUC) samples. We identified 293 genes with >=3 or <=0.3 -fold change. Moreover, there is high overlapping among these three gene lists (20.8-27.4%, FIG. 13C) and the Reactome pathway analysis demonstrated that they are all associated with immune system, innate immune function, and neutrophil degranulation (FIG. 13D).

### BCL2A1 Overexpression Confers Venetoclax Resistance

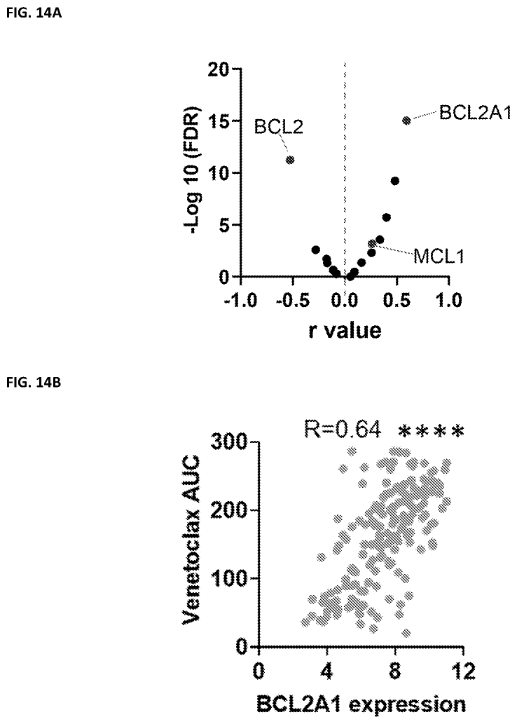

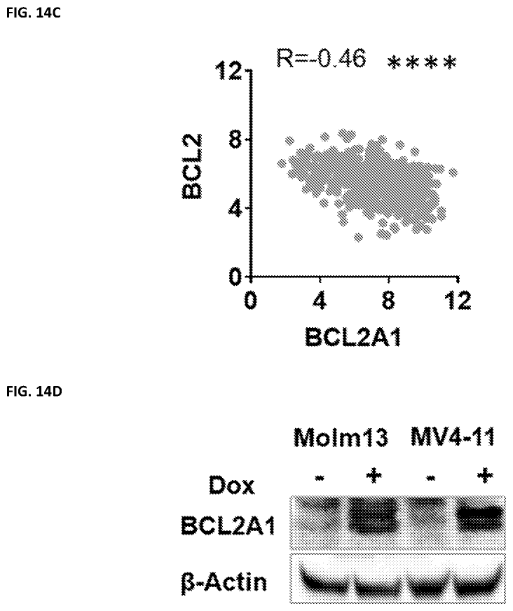

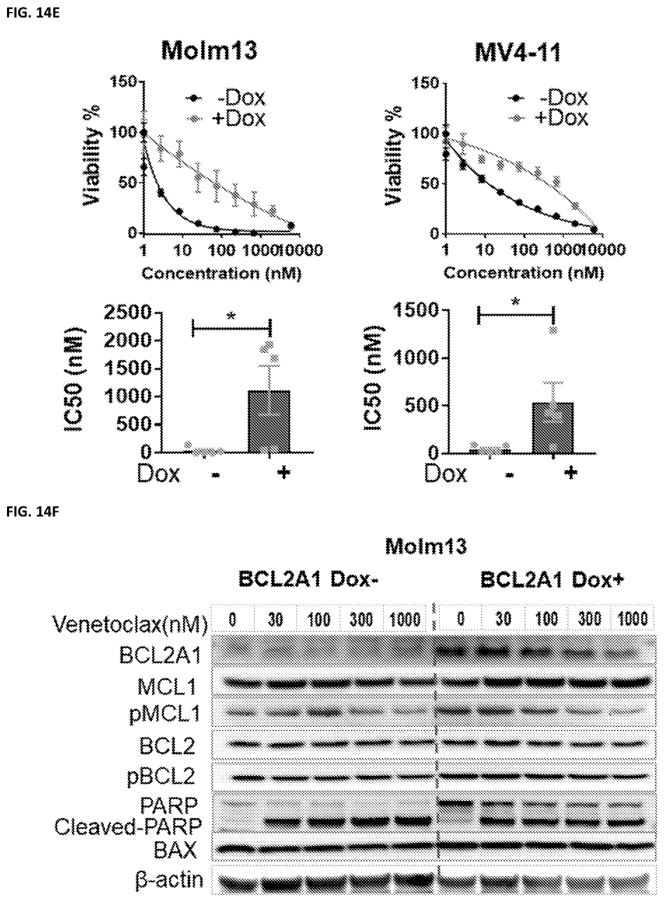

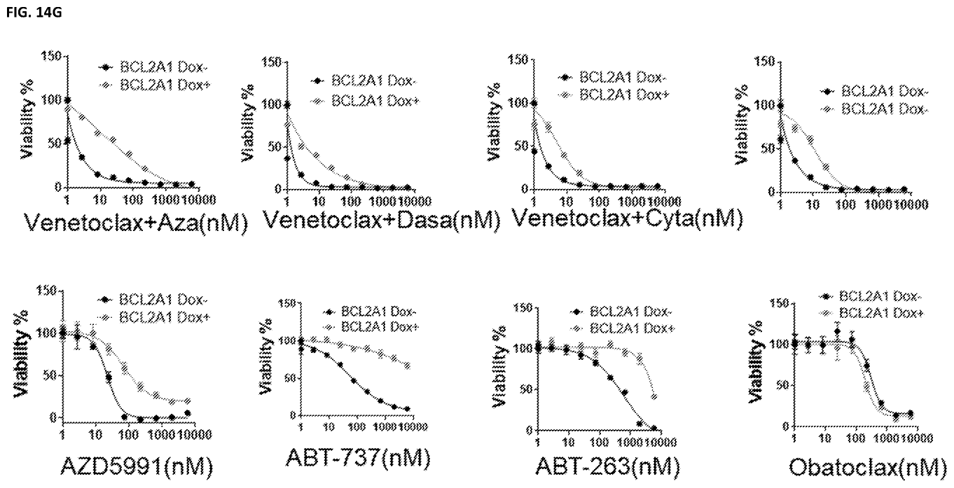

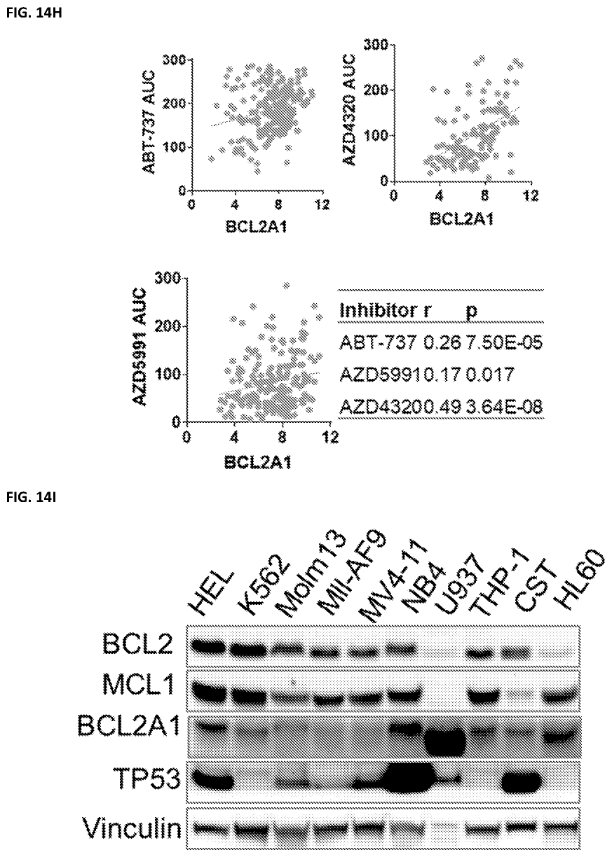

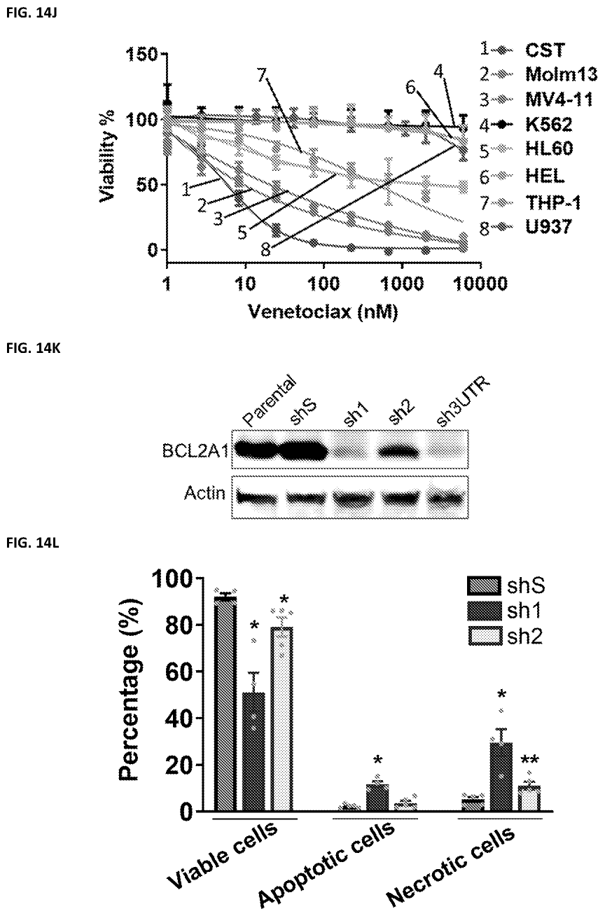

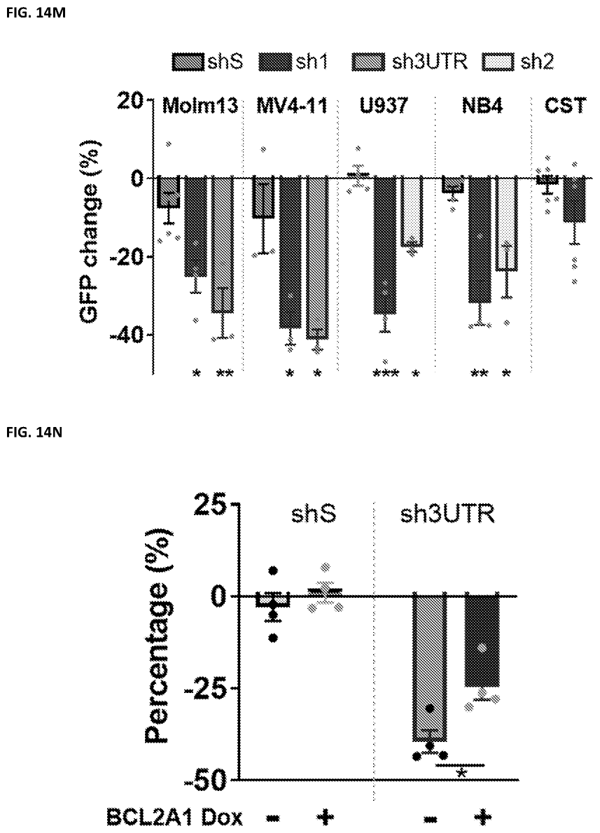

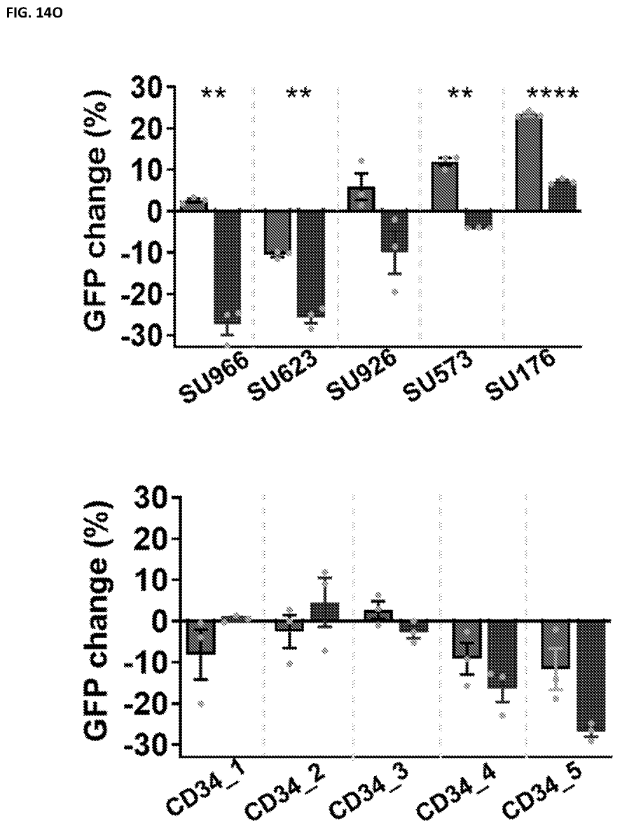

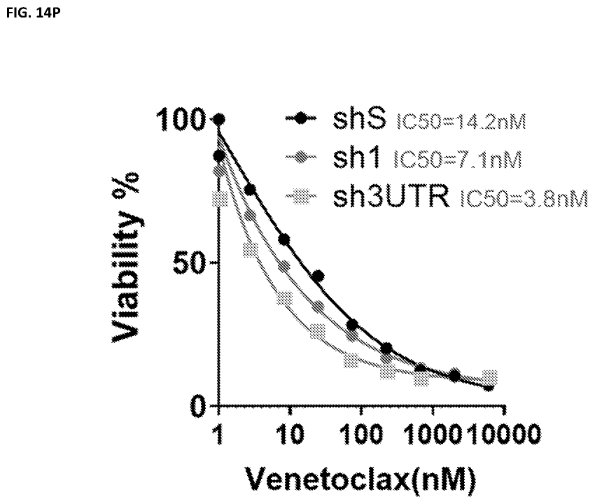

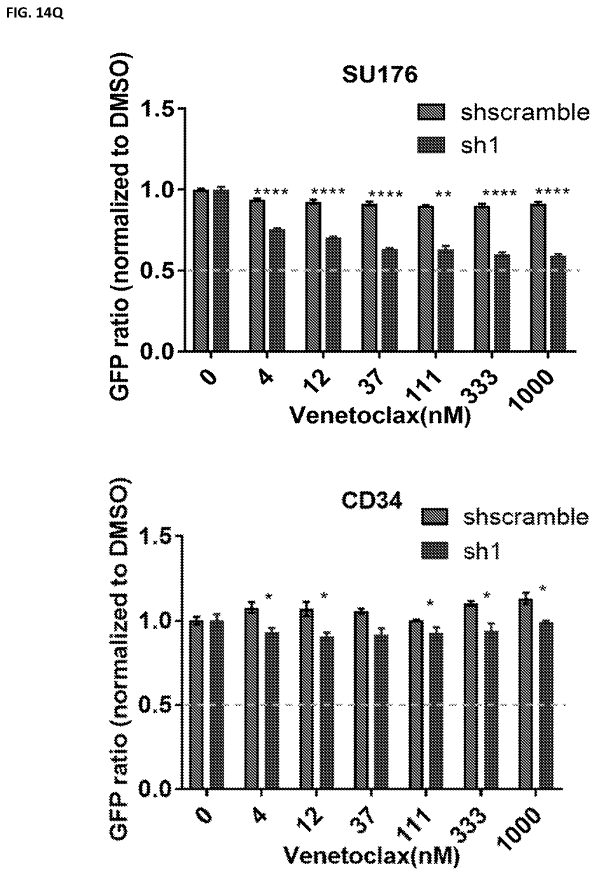

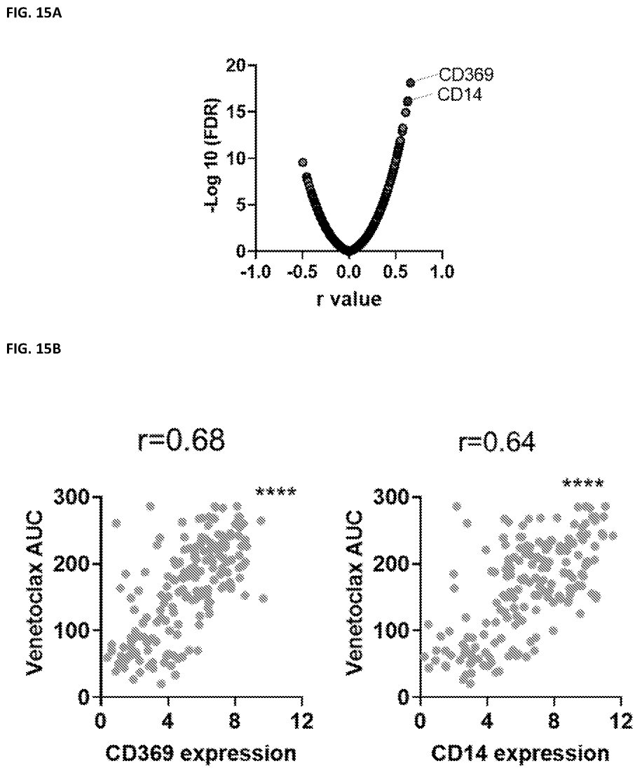

We have identified a number of genes whose expression correlated with venetoclax sensitivity. In order to identify major drivers of venetoclax sensitivity, we first focused on BCL2 family members. As expected, we observed that BCL2 expression was inversely correlated with venetoclax AUC (FIG. 14A). Interestingly, BCL2A1 expression was positively correlated with venetoclax sensitivity, and its correlation was the highest among 17 common BCL2 family genes (FIGS. 14A-B). Moreover, BCL2A1 was in all three lists of genes associated with venetoclax sensitivity. Interestingly, BCL2A1 expression was negatively correlated with BCL2 expression (FIG. 14C). To investigate if BCL2A1 upregulation causes venetoclax resistance, we transduced venetoclax sensitive AML cell lines (Molm13 and MV4-11) with a doxycycline (Dox) inducible BCL2A1 overexpression lentivirus (FIG. 14D). Indeed, we observed that BCL2A1 overexpression induced 39- and 21-fold increase of venetoclax IC50 for Molm13 and MV4-11 respectively (FIG. 14E). While venetoclax readily induced apoptosis in empty vector (Dox−) cells, only marginal apoptosis occurred in BCL2A1 overexpression cells (Dox+), even at a high drug concentration (1000 nM), as indicated by PARP cleavage (FIG. 14N). Given that expression of other anti-apoptotic BCL2 family proteins did not change between Dox+ and Dox− cells (FIG. 14N), the BCL2A1-mediated venetoclax resistance phenotype is likely to be an on-target and direct effect of BCL2A1 alone. Furthermore, from cell line models and primary AML inhibitor sensitivity assay, we observed that BCL2A1 overexpression also conferred relative resistance to venetoclax combinations and the majority of other BCL2 family inhibitors in the ex vivo drug assay, but not azacytidine/cytarabine alone, or the pan BCL2 family inhibitor obatoclax, which also targets BCL2A1 (FIGS. 14O-Q and and 20A-C).

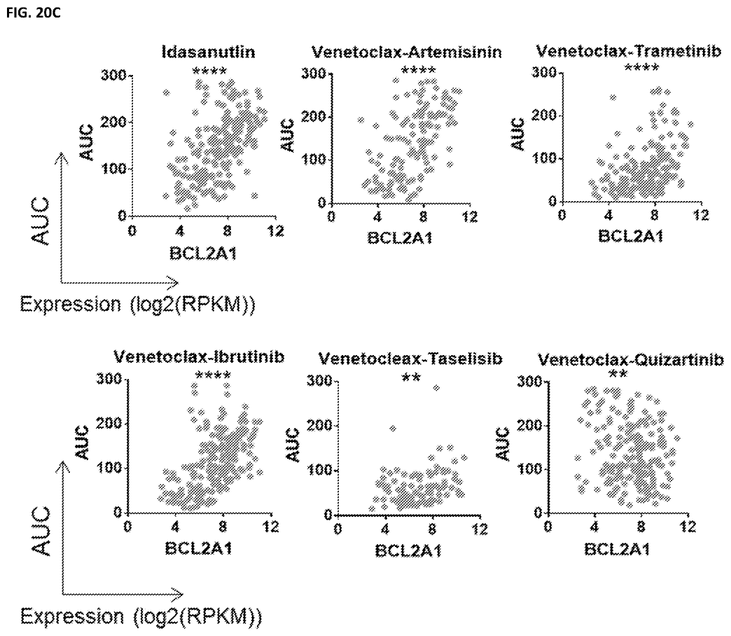

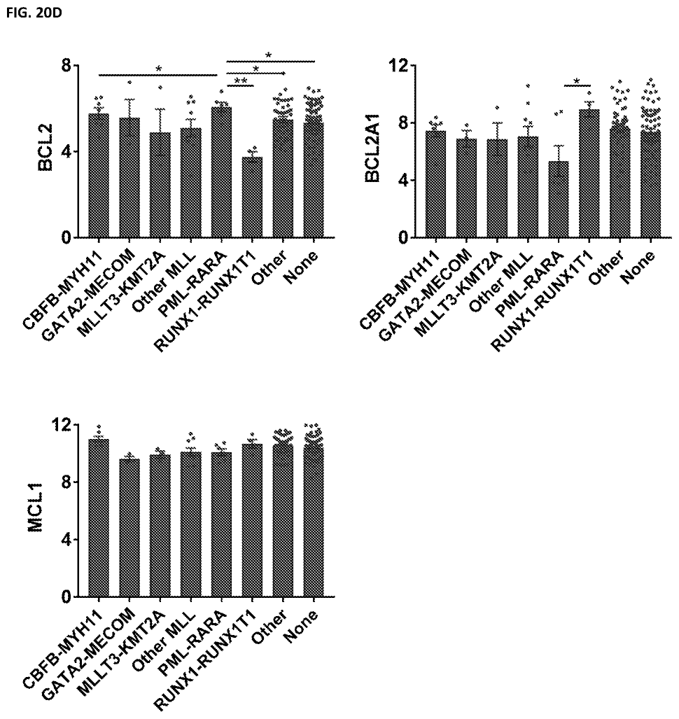

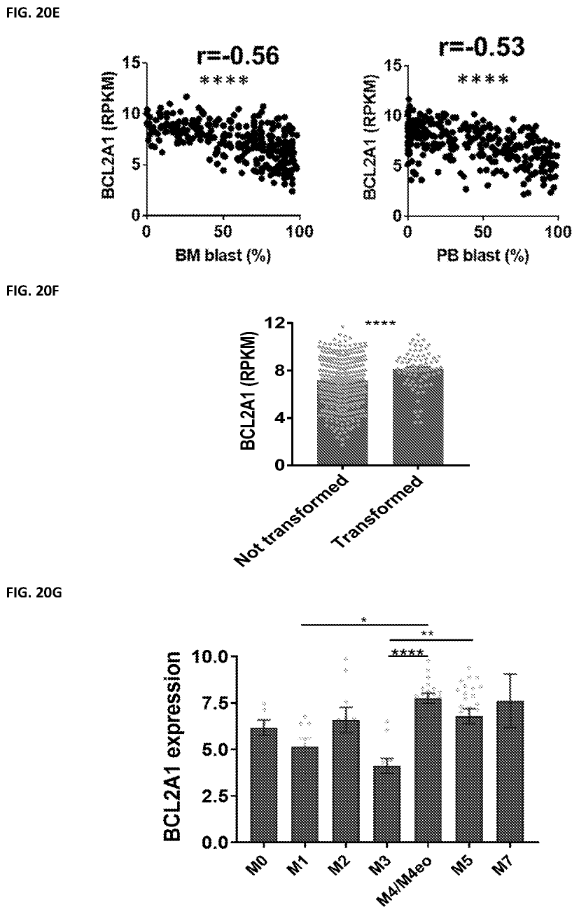

Next, we compared the expression of BCL2A1, BCL2, and MCL1 among different leukemia subsets by mining previous large cohort gene expression datasets. We observed that CLL samples demonstrated higher BCL2, lower BCL2A1, and a similar level of MCL1 expression compared to AML or acute lymphoblastic leukemia. This may explain the high sensitivity of venetoclax in CLL samples and the low response rate of venetoclax alone in AML samples. Accordingly, AML samples with PML-RARA fusion that were venetoclax sensitive also demonstrated relatively high BCL2, low BCL2A1, and similar MCL1 expression compared to other subsets of AML samples (FIG. 20D). In contrast, AML samples with low blast count, a high neutrophil count, PML-RARA fusion, FAB M4/M5, and/or transformed AML that were venetoclax sensitive also demonstrated relatively high BCL2A1 expression (20F-H).

### Targeting BCL2A1 Induces Apoptosis, Inhibits Proliferation, and Synergizes with Venetoclax

In normal hematopoiesis, BCL2A1 is expressed at a low level in hematopoietic stem cells (HSC), and at a high level in mature monocytes and granulocytes. We also observed that BCL2A1 was upregulated in the majority, MCL1 in more than half, and BCL2 in a small subset of AML samples. Moreover, previous studies showed that BCL2A1 knockout mice are viable, fertile, and demonstrate minimal defects in HSC function and myeloid cell differentiation³¹. This prompted us to further investigate whether knockdown of BCL2A1 has an anti-leukemia effect, while sparing normal hematopoiesis. We observed that AML cell lines express various levels of BCL2A1, which partially correlated with venetoclax sensitivity (FIG. 14I). Knockdown of BCL2A1 with shRNA (sh1, sh2, and sh3UTR), but not the scramble control (shS) induced enhanced apoptosis in U937 cells and inhibited cell proliferation in AML cell lines (FIGS. 14K-M). The anti-proliferation effect could be partially rescued by overexpression of BCL2A1 (FIG. 14F), indicating an on-target effect. Notably, knockdown of BCL2A1 also inhibited proliferation of primary AML cells, but not CD34+ cord blood hematopoietic stem and progenitor cell (HSPC) controls (FIG. 14G). Consistent with data from BCL2A1 knockout mice, knockdown of BCL2A1 did not affect CD34+ HSPC colony-forming ability³¹, indicating a desirable therapeutic window to target BCL2A1 upregulated leukemia cells while sparing healthy HSPCs. Furthermore, knockdown of BCL2A1 sensitized Molm13 cells and primary AML cells to venetoclax, but not CD34+ HSPCs (FIGS. 14H-I).

### Expression of CD369 or CD14 Predicts Venetoclax Resistance

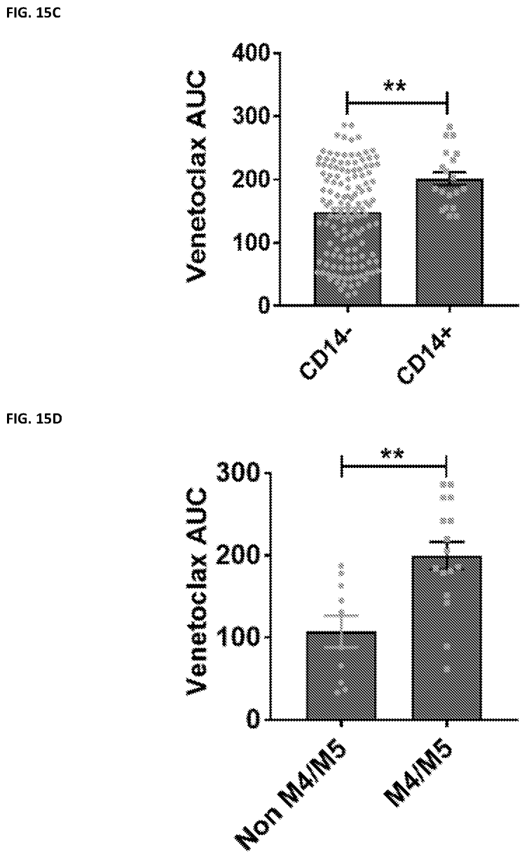

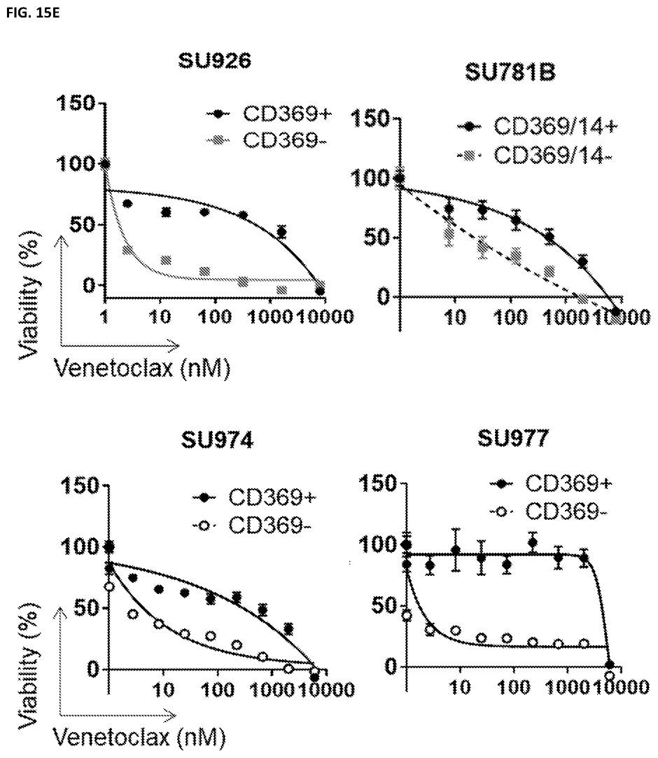

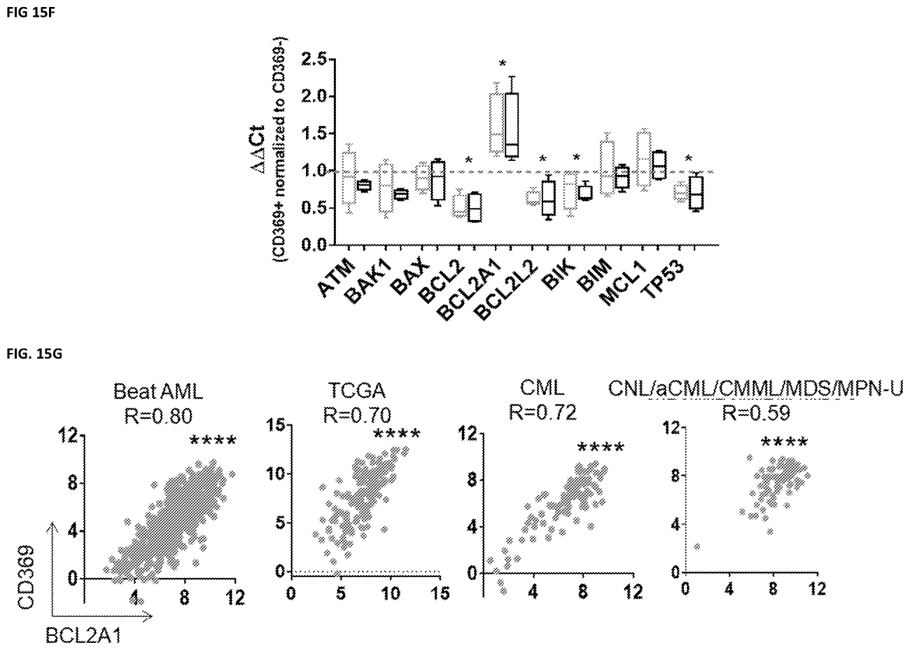

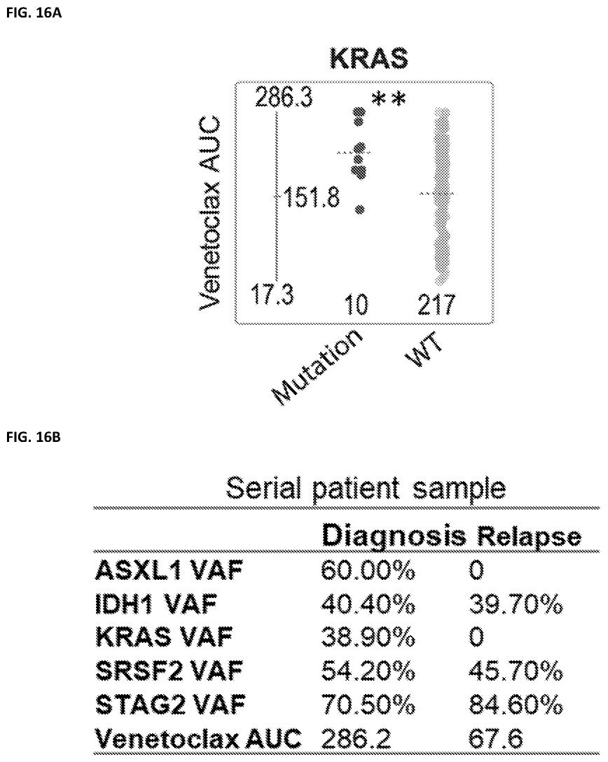

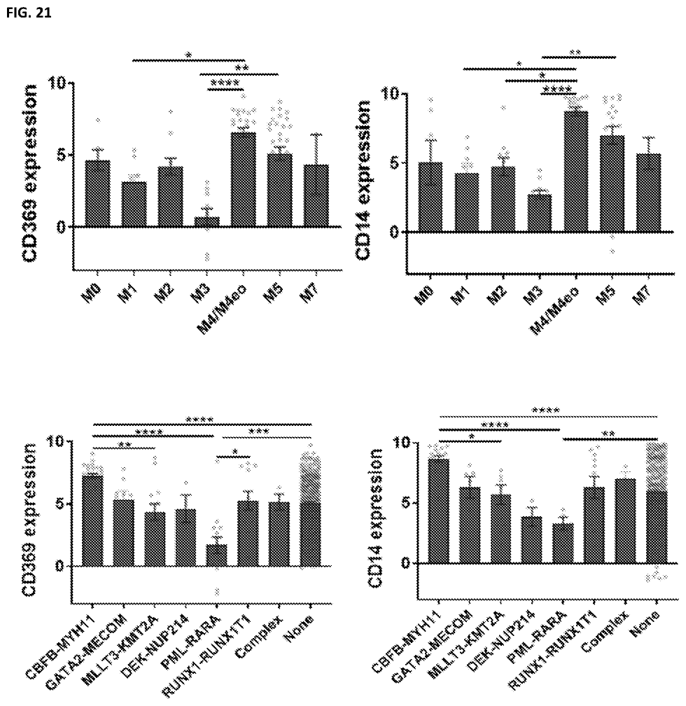

To identify clinically applicable cell surface markers that could be used as biomarkers for venetoclax sensitivity and/or BCL2A1 upregulation, we screened the correlation between venetoclax and cell surface Gene Ontology (GO) Term related genes. The top two genes were CD369 and CD14, which are both monocyte and mature neutrophil cell surface markers that are expressed at low levels in normal HSPCs as shown by multiple large cohort RNAseq analysis (FIGS. 15A-B). Similar to CD14, CD369 is also expressed at higher levels in AML M4 and M5 subsets, and at a lower lever in AML with PML-RARA or M3 (FIG. 21). Accordingly, leukemia blasts with CD14 expression demonstrated higher venetoclax AUCs compared to leukemia blasts with no CD14 expression determined by clinical immunophenotyping in the Beat AML cohort. Furthermore, AML FAB M4/M5 subgroups showed higher venetoclax AUC comparing to FAB non-M4/M5 subsets (FIG. 15D).

To further validate that CD369 and CD14 surface expression is associated with venetoclax resistance, we sorted leukemic blasts into CD369 and/or CD14 positive and negative cells and performed a venetoclax sensitivity assay. CD369 and/or CD14+ blasts demonstrated higher venetoclax AUC compared to CD369 and/or CD14− blasts in all 4 tested primary AML samples (FIG. 15E). Furthermore, CD369 and/or CD14+ cells expressed higher BCL2A1, and lower BCL2 and TP53 compared to the marker negative blast cells (FIG. 15F). Notably, CD369 and CD14 expression correlated with BCL2A1 expression from Beat AML, AML TCGA³, and internal CML (paper in preparation) and CNL cohorts³² (FIG. 15G). These data indicate that high expression of CD369 and CD14 is associated with venetoclax resistance, likely due in part to high expression of BCL2A1 in these cells.

### KRAS Mutations Confer Venetoclax Resistance

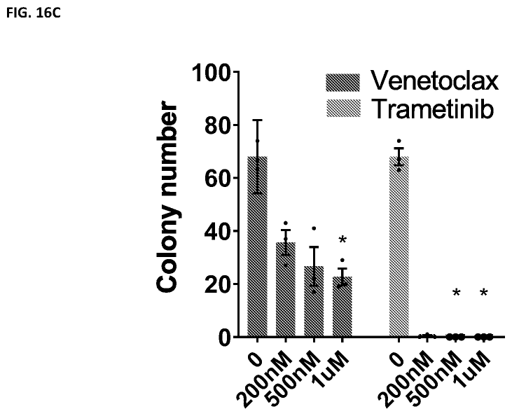

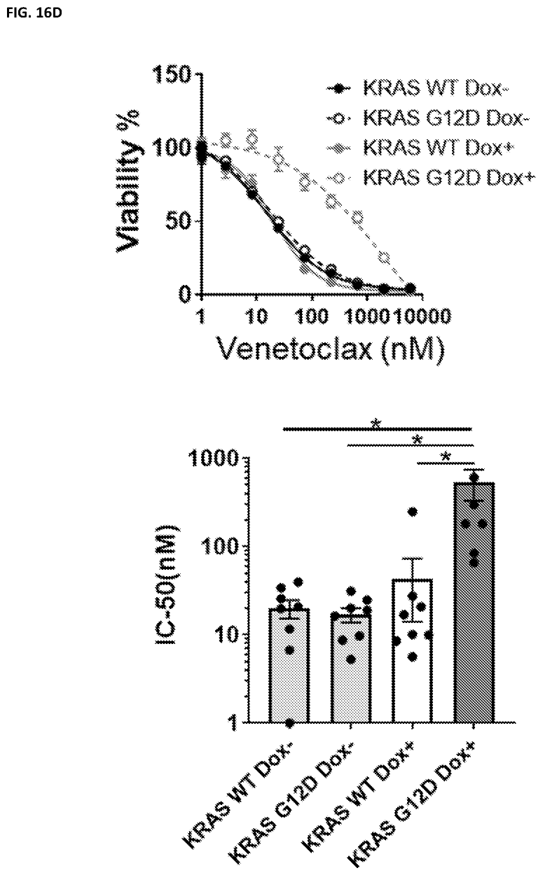

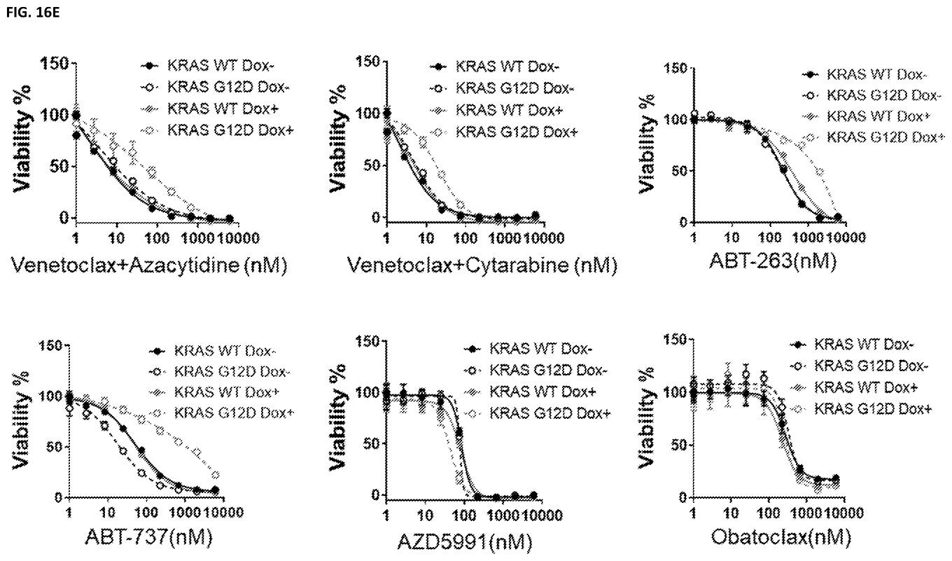

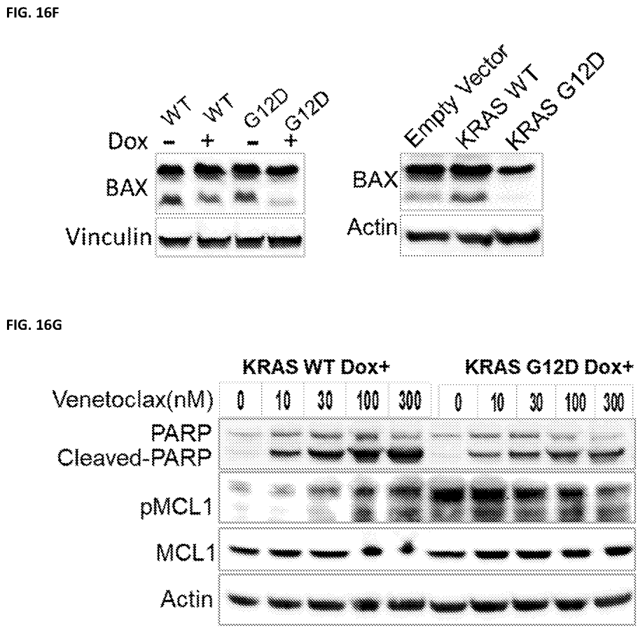

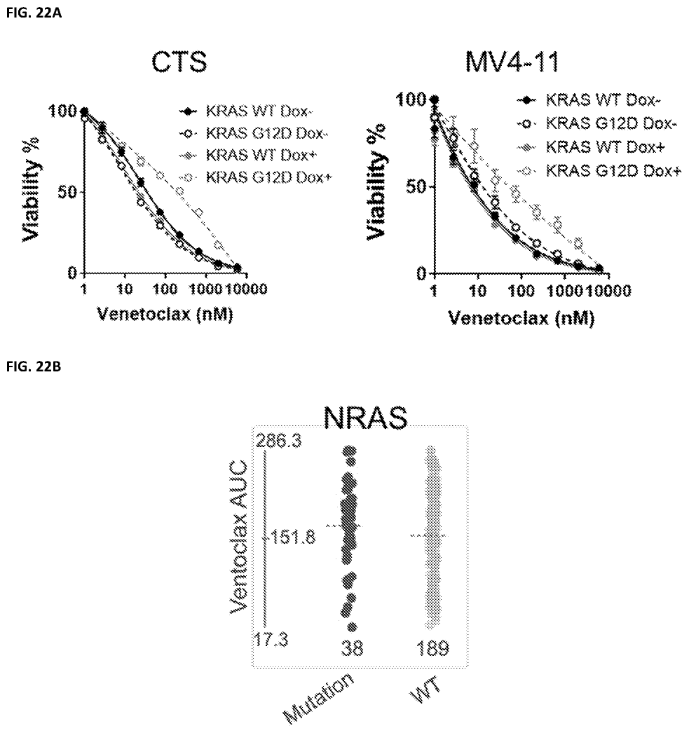

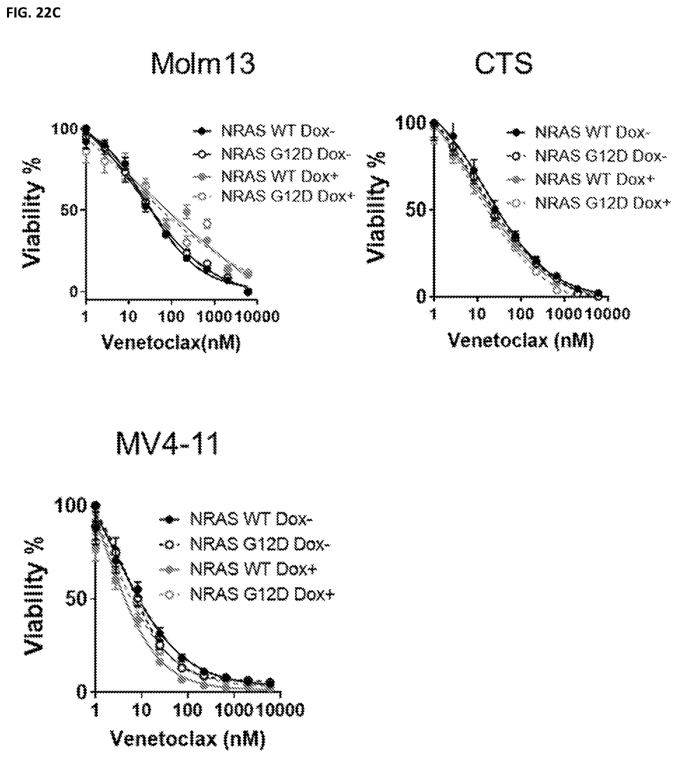

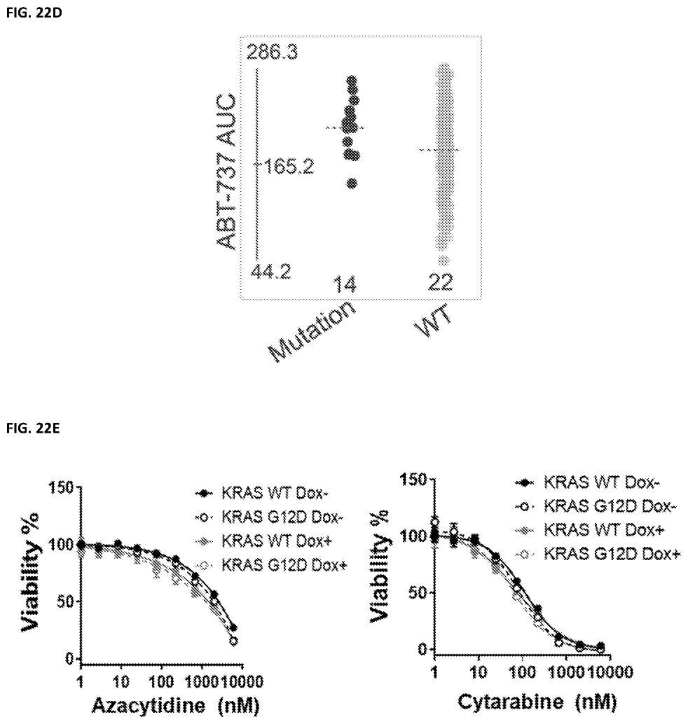

From the Beat AML cohort, we also observed that KRAS mutations demonstrated higher venetoclax AUC compared to KRAS wild type (WT) samples (FIGS. 13B and 16A). Interestingly, one AML patient sample was venetoclax resistant before chemotherapy with KRAS, IDH1, ASXL1, SRSF2, and STAG2 mutations, but became venetoclax sensitive at disease relapse with the loss of KRAS and ASXL1 mutations (FIG. 16B). To further determine if the KRAS mutation, but not the co-occurring mutations, accounts for resistance to venetoclax, we first overexpressed mutant KRAS in mouse bone marrow (BM) lineage-negative stem and progenitor cells. We observed that KRAS G12D-expressing cells were relatively resistant to venetoclax in comparison to trametinib, a MEK inhibitor (FIG. 16C). We also overexpressed KRAS WT and G12D with a Dox-inducible lentivirus in three different AML cell lines. We observed that KRAS G12D, but not KRAS WT, induced venetoclax resistance (FIGS. 16D and 22A). Interestingly, NRAS G12D did not confer venetoclax resistance as shown from the primary patient data and from our cell line models (FIGS. 22B-C). Furthermore, KRAS G12D also conferred resistance to venetoclax combinations and two BCL2, BCL-w, and BCL-xL inhibitors (ABT-263 and ABT-737), but not an MCL1 inhibitor AZD5991, the pan-BCL2 family inhibitor obatoclax, or other single agents (FIGS. 16E and 22D-E).

To explore the potential underlying mechanisms associated with KRAS mutant-mediated venetoclax resistance, we performed immunoblot analysis. We observed that KRAS G12D-expressing cells demonstrated reduced BAX expression and sustained phosphorylation of MCL1 (pMCL1), which have been shown to be associated with resistance to apoptosis (FIGS. 16F-G)³³⁻³⁵. We did not observe obvious changes in other KRAS or apoptosis-related proteins. These data, together with the inhibitor data showing that KRAS mutant cells remained sensitive to MCL1 inhibition, suggest that KRAS mutations induce venetoclax resistance, at least partially through upregulation of pMCL1.

### PTPN11 Mutations Confer Venetoclax Resistance

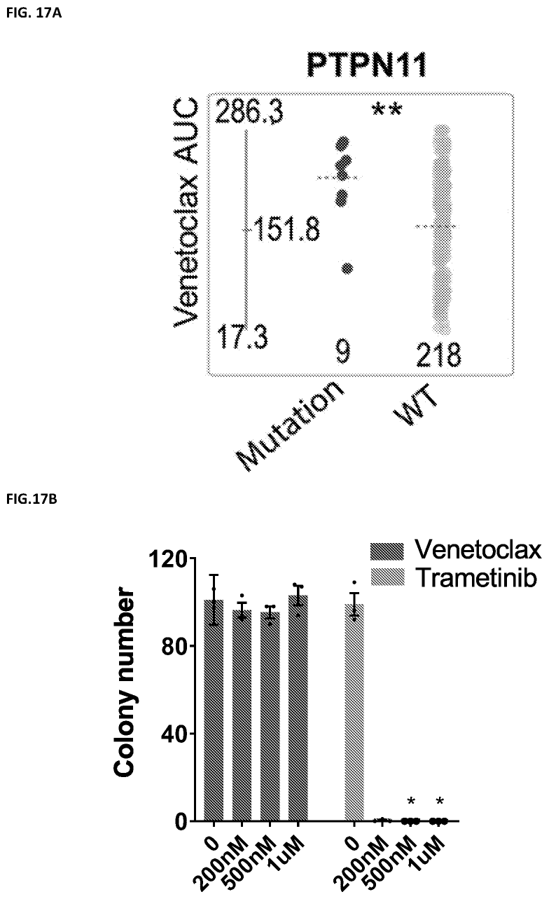

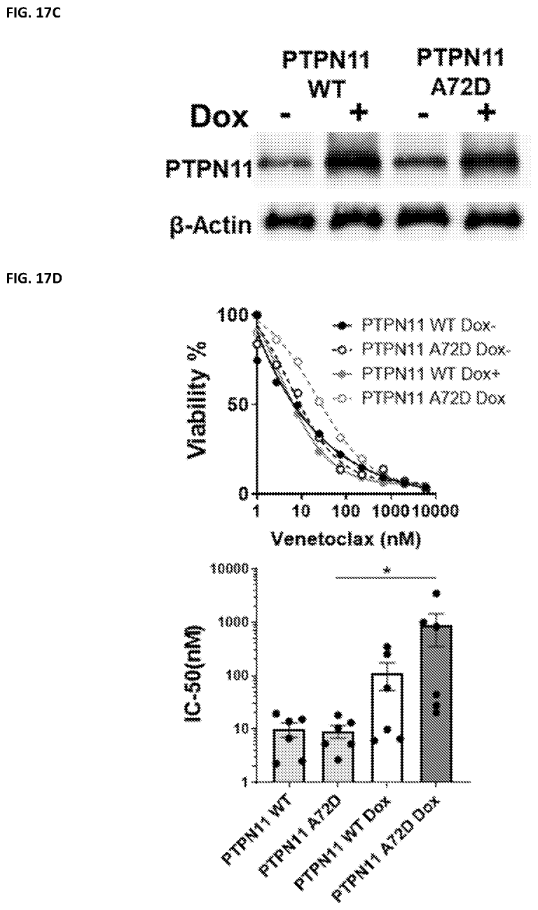

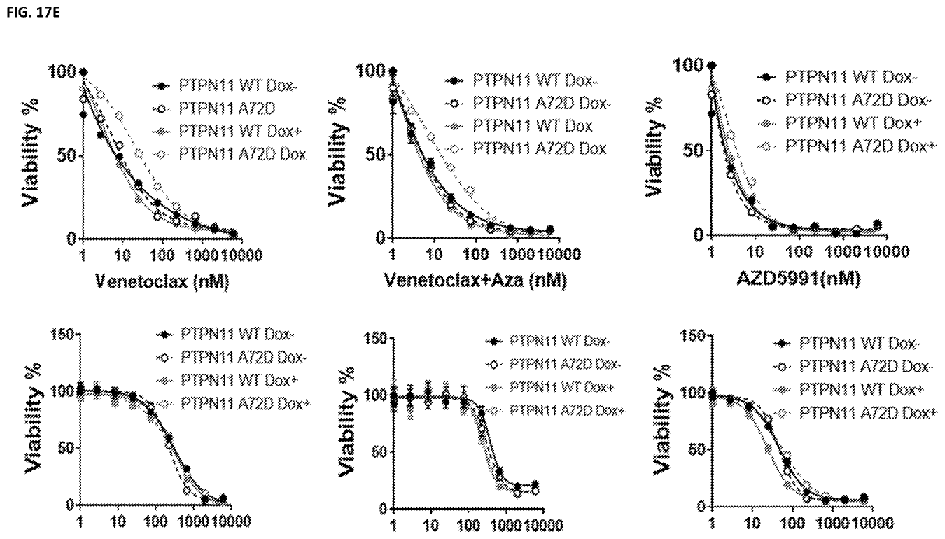

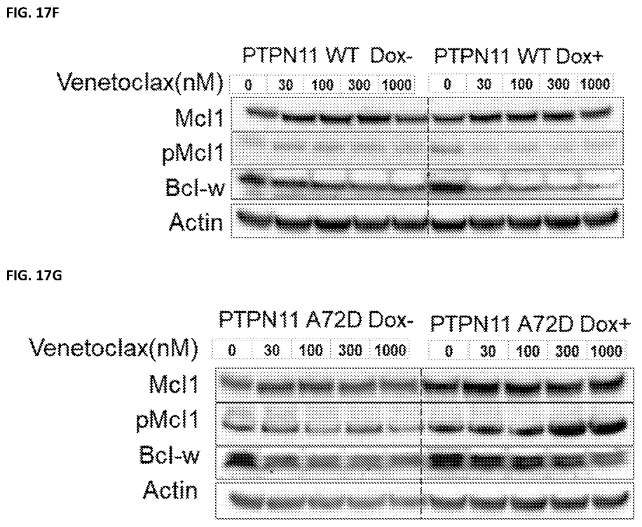

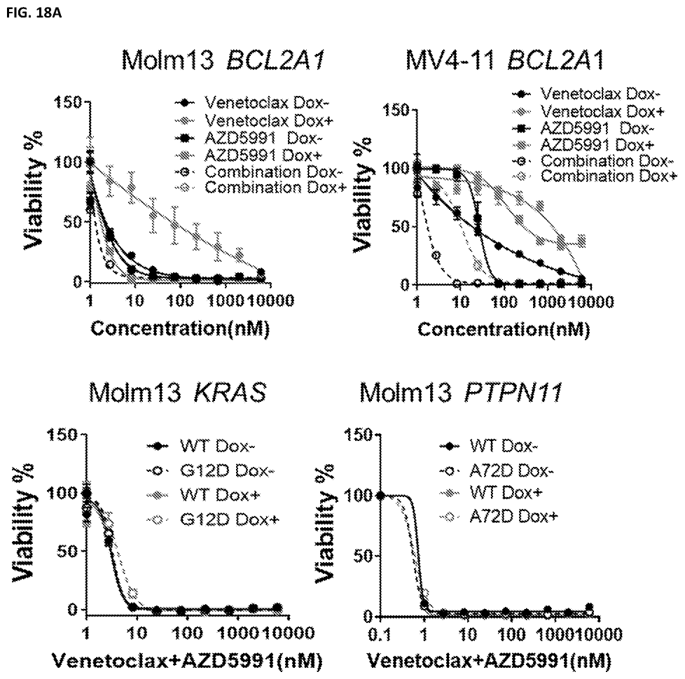

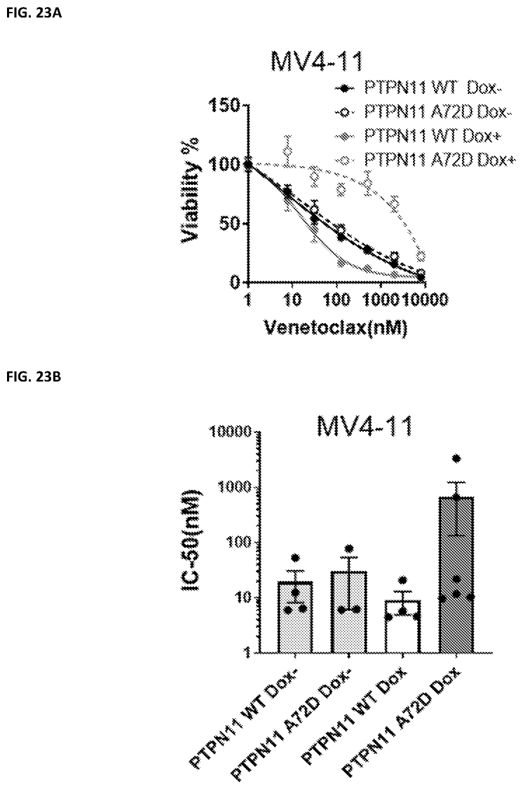

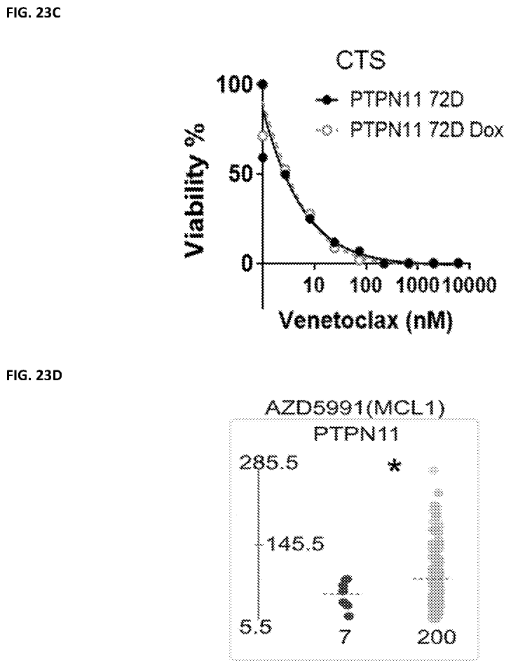

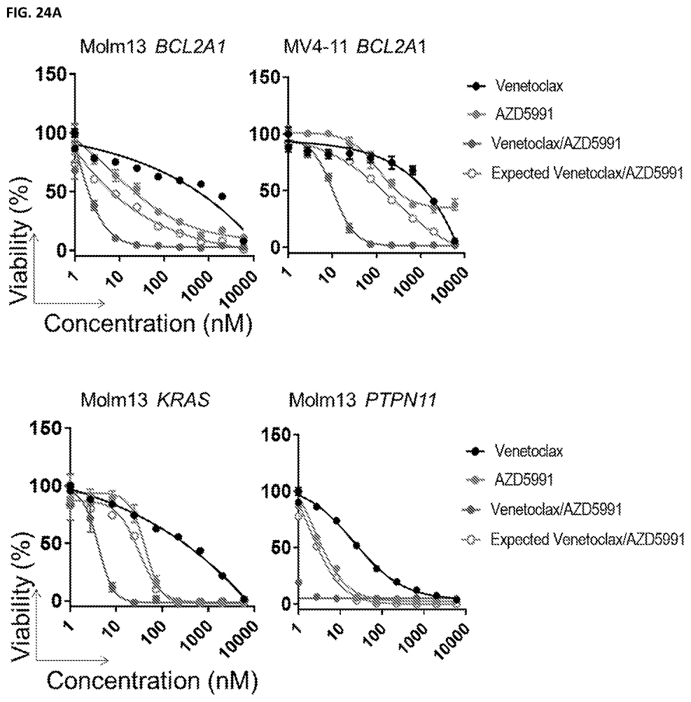

To investigate if PTPN11 mutations alone are sufficient to induce the venetoclax resistance observed in the Beat AML cohort (FIGS. 13B and 17A), we overexpressed mutant PTPN11 in mouse BM lineage-negative HSPCs and three human AML cell lines (FIGS. 17B-C). We observed that PTPN11 A72D was relatively resistant to venetoclax compared to trametinib and induced venetoclax resistance upon inducible expression in Molm13 and MV4-11 cells, but not CTS cells, which express a low level of MCL1 (FIGS. 17B-D and 23A-C). Furthermore, PTPN11 A72D showed resistance to venetoclax combinations, but not other BCL2 family inhibitors from both our cell line models and the Beat AML primary patient screening assay (FIGS. 17D and 23D).

We further performed BCL2 protein immunoblotting to understand the mechanisms associated with mutant-PTPN11 mediated venetoclax resistance. We observed that PTPN11 A72D induced enhanced and sustained pMCL1 and BCL-w expression in the presence or absence of venetoclax in Molm13 and MV4-11 cells, but not CTS cells (FIG. 17F). We did not observe obvious changes in other PTPN11 or apoptosis-related proteins. These data suggest that mutant-PTPN11 mediated venetoclax resistance is at least partially dependent on increased pMCL1 and BCL-w, which could be targeted by the MCL1 inhibitor AZD5991 and BCL-w inhibitors ABT-263 and ABT-737.

Provided is a method of treating a venetoclax-resistant Acute Myeloid Leukemia in a human subject, wherein a high level of expression of pMCL1 is present, the method comprising administering to the human subject in need thereof a therapeutically effective amount of AZD5991, or a pharmaceutically acceptable salt thereof, and a therapeutically effective amount of venetoclax, or a pharmaceutically acceptable salt thereof.

Also provided is a method of treating a venetoclax-resistant Acute Myeloid Leukemia in a human subject, wherein a high level of expression of BCL-w is present, the method comprising administering to the human subject in need thereof a therapeutically effective amount of a BCL-w inhibitor, or a pharmaceutically acceptable salt thereof, and a therapeutically effective amount of venetoclax, or a pharmaceutically acceptable salt thereof.

Provided is a method of treating a venetoclax-resistant Acute Myeloid Leukemia in a human subject, wherein a high level of expression of both pMCL1 and BCL-w is present, the method comprising administering to the human subject in need thereof:
a) a therapeutically effective amount of AZD5991, or a pharmaceutically acceptable salt thereof;
b) a therapeutically effective amount of a BCL-w inhibitor, or a pharmaceutically acceptable salt thereof; and
c) a therapeutically effective amount of venetoclax, or a pharmaceutically acceptable salt thereof

Also provided is a method of diagnosing and treating venetoclax-resistant Acute Myeloid Leukemia in a human subject, the method comprising:
a) obtaining a biological sample from the human subject;
b) detecting whether a high level of expression of pMCL1 is present in the biological sample;
c) diagnosing the human subject with venetoclax-resistant Acute Myeloid Leukemia when a high level of expression of pMCL1 is detected in the biological sample; and
d) administering to the human subject in need thereof a pharmaceutically effective amount of venetoclax, or a pharmaceutically acceptable salt thereof, and a pharmaceutically effective amount of AZD5991, or a pharmaceutically acceptable salt thereof.

Further provided is a method of diagnosing and treating venetoclax-resistant Acute Myeloid Leukemia in a human subject, the method comprising:
e) obtaining a biological sample from the human subject;
f) detecting whether a high level of expression of BCL-w is present in the biological sample;
g) diagnosing the human subject with venetoclax-resistant Acute Myeloid Leukemia when a high level of expression of BCL-w is detected in the biological sample; and
h) administering to the human subject in need thereof a pharmaceutically effective amount of venetoclax, or a pharmaceutically acceptable salt thereof, and a pharmaceutically effective amount of a BCL-w inhibitor, or a pharmaceutically acceptable salt thereof.

Further provided is a method of diagnosing and treating venetoclax-resistant Acute Myeloid Leukemia in a human subject, the method comprising:
a) obtaining a biological sample from the human subject;
b) detecting whether a high level of expression of BCL-w and a high level of expression of pMCL1 are each present in the biological sample;
c) diagnosing the human subject with venetoclax-resistant Acute Myeloid Leukemia when a high level of expression of pMCL1 and a high level of expression of pMCL1 are each present in the biological sample is detected in the biological sample; and
d) administering to the human subject in need thereof:
   i) a pharmaceutically effective amount of venetoclax, or a pharmaceutically acceptable salt thereof;
   ii) a pharmaceutically effective amount of a BCL-w inhibitor, or a pharmaceutically acceptable salt thereof; and
   iii) a pharmaceutically effective amount of AZD5991, or a pharmaceutically acceptable salt thereof.

In some embodiments of the methods herein, the BCL-w inhibitor is selected from the group of Navitoclax (ABT-263) and ABT-737.

Also provided is a method of treating a venetoclax-resistant Acute Myeloid Leukemia in a human subject, wherein the Acute Myeloid Leukemia is characterized by BCL2A1 overexpression, the method comprising administering to the human subject in need thereof a therapeutically effective amount of AZD5991, or a pharmaceutically acceptable salt thereof.

Also provided is a method of treating a venetoclax-resistant Acute Myeloid Leukemia in a human subject, wherein the Acute Myeloid Leukemia is characterized by BCL2A1 overexpression, the method comprising administering to the human subject in need thereof a therapeutically effective amount of AZD5991, or a pharmaceutically acceptable salt thereof, and a therapeutically effective amount of venetoclax, or a pharmaceutically acceptable salt thereof.

Also provided is a method of treating a venetoclax-resistant Acute Myeloid Leukemia in a human subject, wherein the Acute Myeloid Leukemia is characterized by a KRAS mutation, the method comprising administering to the human subject in need thereof a therapeutically effective amount of AZD5991, or a pharmaceutically acceptable salt thereof. Another embodiment provides a method of treating a venetoclax-resistant Acute Myeloid Leukemia in a human subject, wherein the Acute Myeloid Leukemia is characterized by a KRAS mutation, the method comprising administering to the human subject in need thereof a therapeutically effective amount of AZD5991, or a pharmaceutically acceptable salt thereof, and a therapeutically effective amount of venetoclax, or a pharmaceutically acceptable salt thereof. In some embodiments, the KRAS mutation is a KRAS G12D mutation.

Also provided is a method of treating a venetoclax-resistant Acute Myeloid Leukemia in a human subject, wherein the Acute Myeloid Leukemia is characterized by a PTPN11 mutation, the method comprising administering to the human subject in need thereof a therapeutically effective amount of AZD5991, or a pharmaceutically acceptable salt thereof. Another embodiment provides a method of treating a venetoclax-resistant Acute Myeloid Leukemia in a human subject, wherein the Acute Myeloid Leukemia is characterized by a PTPN11 mutation, the method comprising administering to the human subject in need thereof a therapeutically effective amount of AZD5991, or a pharmaceutically acceptable salt thereof, and a therapeutically effective amount of venetoclax, or a pharmaceutically acceptable salt thereof. In some embodiments, the PTPN11 mutation is a PTPN11 A72D mutation.

Also provided is a method of diagnosing and treating venetoclax-resistant Acute Myeloid Leukemia in a human subject, the method comprising:
a) obtaining a biological sample from the human subject;
b) detecting whether BCL2A1 overexpression is present in the biological sample;
c) diagnosing the human subject with venetoclax-resistant Acute Myeloid Leukemia when BCL2A1 overexpression is detected in the biological sample; and
d) administering to the human subject in need thereof a pharmaceutically effective amount of AZD5991, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether BCL2A1 overexpression is present in the biological sample;
c) diagnosing the human subject with venetoclax-resistant Acute Myeloid Leukemia when BCL2A1 overexpression is detected in the biological sample; and
d) administering to the human subject in need thereof a pharmaceutically effective amount of venetoclax, or a pharmaceutically acceptable salt thereof, and a pharmaceutically effective amount of AZD5991, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether a KRAS mutation is present in the biological sample;
c) diagnosing the human subject with venetoclax-resistant Acute Myeloid Leukemia when a KRAS mutation is detected in the biological sample; and
d) administering to the human subject in need thereof a pharmaceutically effective amount of AZD5991, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether a KRAS mutation is present in the biological sample;
c) diagnosing the human subject with venetoclax-resistant Acute Myeloid Leukemia when a KRAS mutation is detected in the biological sample; and
d) administering to the human subject in need thereof a pharmaceutically effective amount of venetoclax, or a pharmaceutically acceptable salt thereof, and a pharmaceutically effective amount of AZD5991, or a pharmaceutically acceptable salt thereof.

Also provided is a method of diagnosing and treating venetoclax-resistant Acute Myeloid Leukemia in a human subject, the method comprising:
a) obtaining a biological sample from the human subject;
b) detecting whether a PTPN11 mutation is present in the biological sample;
c) diagnosing the human subject with venetoclax-resistant Acute Myeloid Leukemia when a PTPN11 mutation is detected in the biological sample; and
d) administering to the human subject in need thereof a pharmaceutically effective amount of AZD5991, or a pharmaceutically acceptable salt thereof.

Also provided is a method of diagnosing and treating venetoclax-resistant Acute Myeloid Leukemia in a human subject, the method comprising:
e) obtaining a biological sample from the human subject;
f) detecting whether a PTPN11 mutation is present in the biological sample;
g) diagnosing the human subject with venetoclax-resistant Acute Myeloid Leukemia when a PTPN11 mutation is detected in the biological sample; and
h) administering to the human subject in need thereof a pharmaceutically effective amount of venetoclax, or a pharmaceutically acceptable salt thereof, and a pharmaceutically effective amount of AZD5991, or a pharmaceutically acceptable salt thereof.

### Venetoclax in Combination with the MCL1 Inhibitor AZD5991 Overcomes Venetoclax Resistance by Cleaving BAX

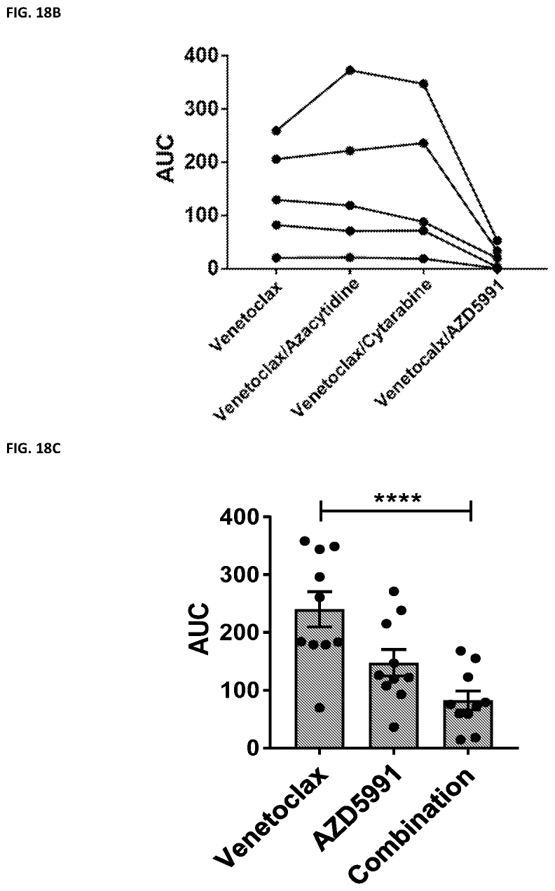

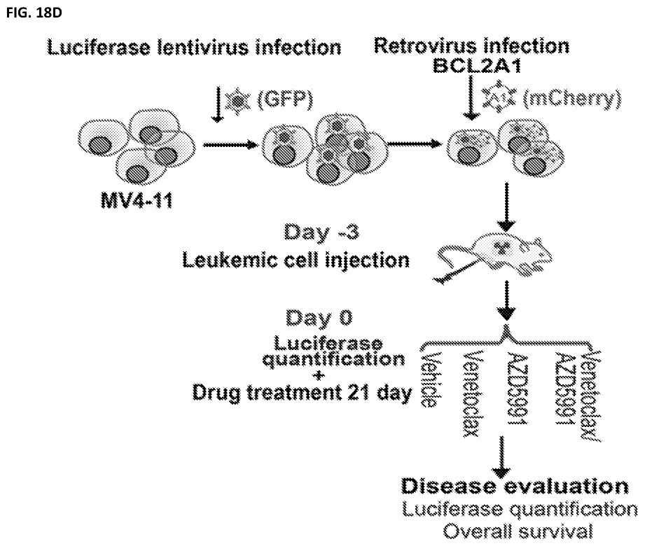

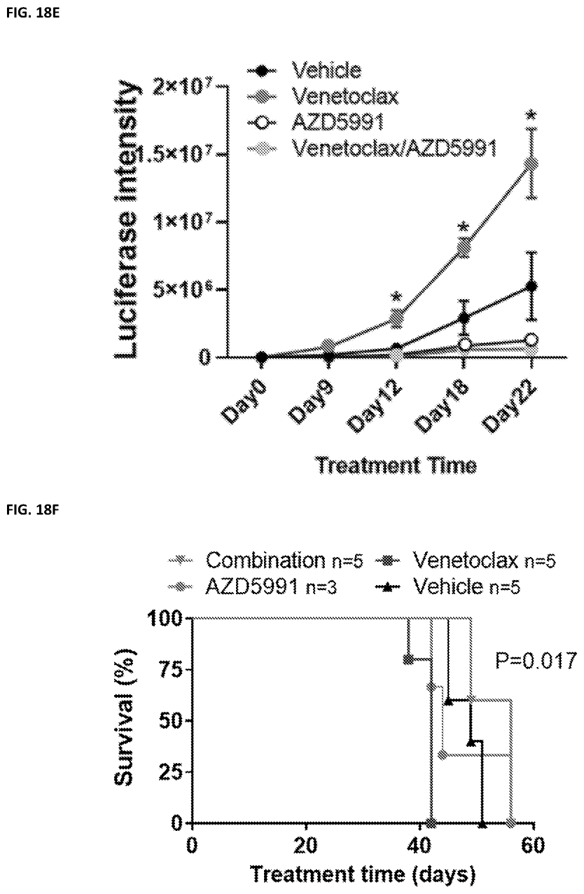

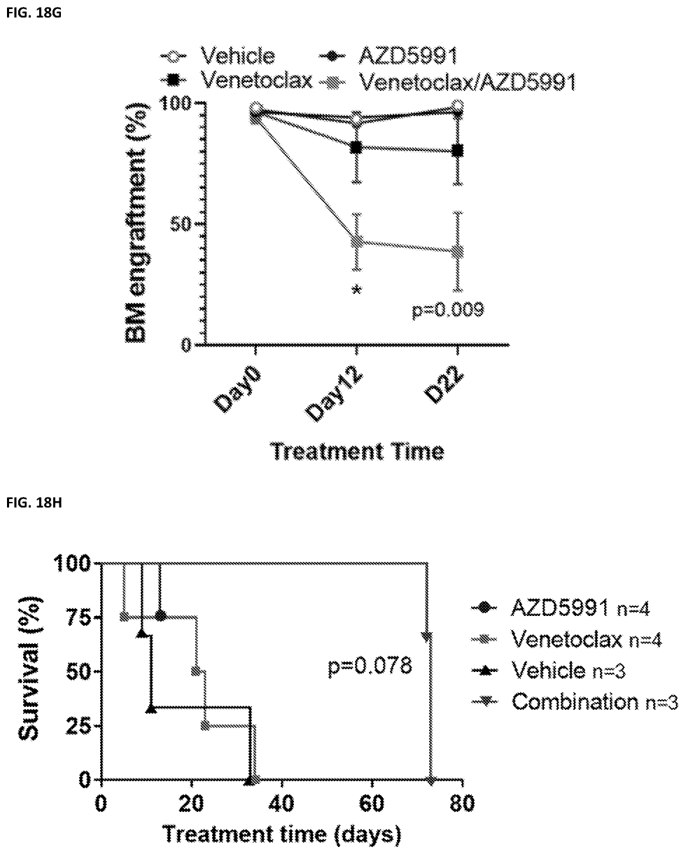

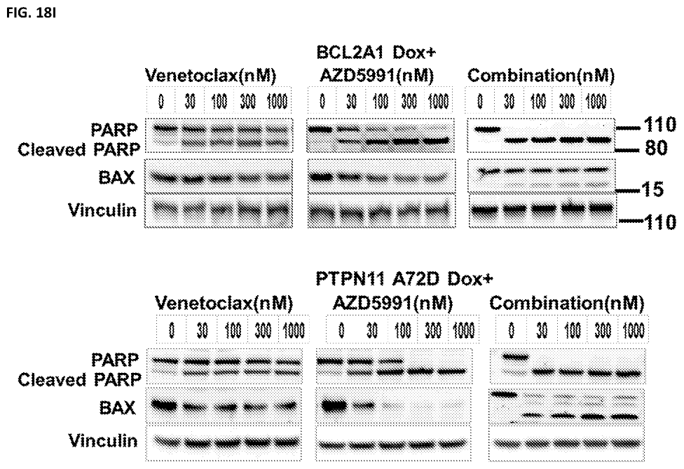

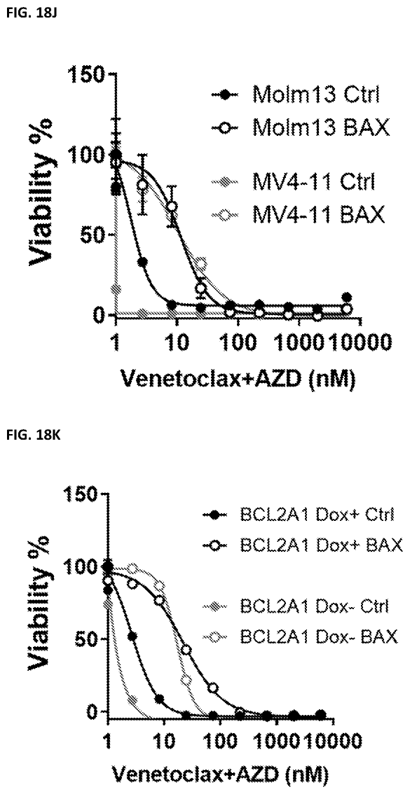

Recent studies have shown that MCL1 inhibitors alone or in combination with venetoclax can overcome venetoclax resistance and are tolerable in human MCL1 knock-in AML xenograft models³⁶⁻³⁸. Therefore, we determined whether venetoclax coupled with the MCL1 inhibitor AZD5991 could circumvent the resistance mediated by BCL2A1 overexpression, KRAS mutations, and PTPN11 mutations. Excitingly, we observed that this combination demonstrated robust cytotoxicity and synergy in all three venetoclax resistant settings, including induction of full sensitivity for KRAS and PTPN11 mutations, and partial rescue of BCL2A1 overexpression-mediated resistance (FIGS. 18A-B and 24A) in cell line models. For primary AML samples, venetoclax in combination with azacytidine or cytarabine remained resistant in the venetoclax-highly resistant samples (AUC>200); whereas venetoclax/AZD5991 combination was effective and synergistic independent of the venetoclax sensitivity status in the majority of the cases (FIGS. 18B-C and 24B-C). To test this in vivo, we overexpressed BCL2A1 in MV4-11 cells expressing luciferase and injected transduced cells into NOD/SCID/gamma (NSG) mice (FIG. 18D). After confirmation of engraftment, the mice were randomly assigned to four treatment groups. We observed that AZD5991 slowed the leukemia progression while the combination significantly reduced the leukemia burden and extended the mice survival (FIGS. 18E-F). We also generated a xenograft model with primary patient leukemia blasts harboring KRAS G12D mutation (FIG. 18G). Strikingly, we observed that the combination dramatically reduced leukemic infiltration in BM and extended the mice survival (FIGS. 18H and 18F).

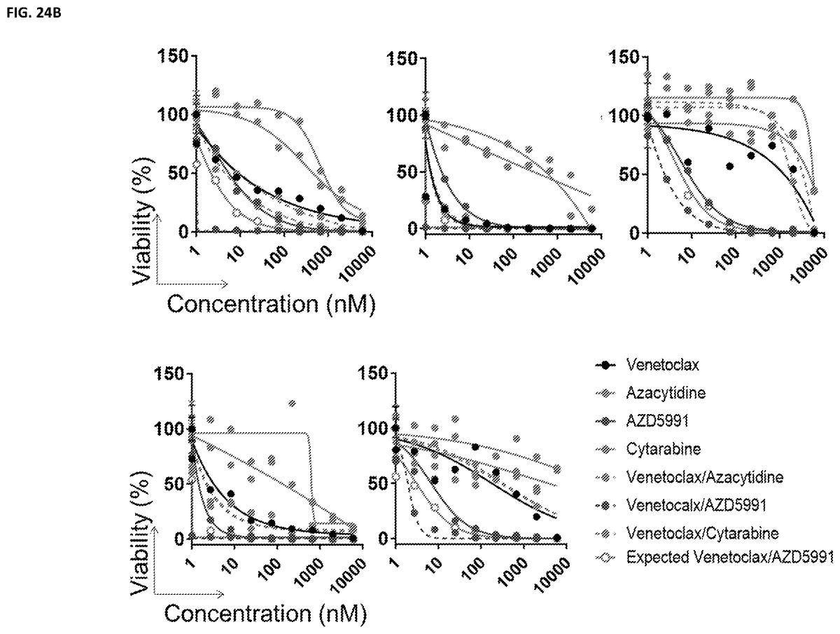

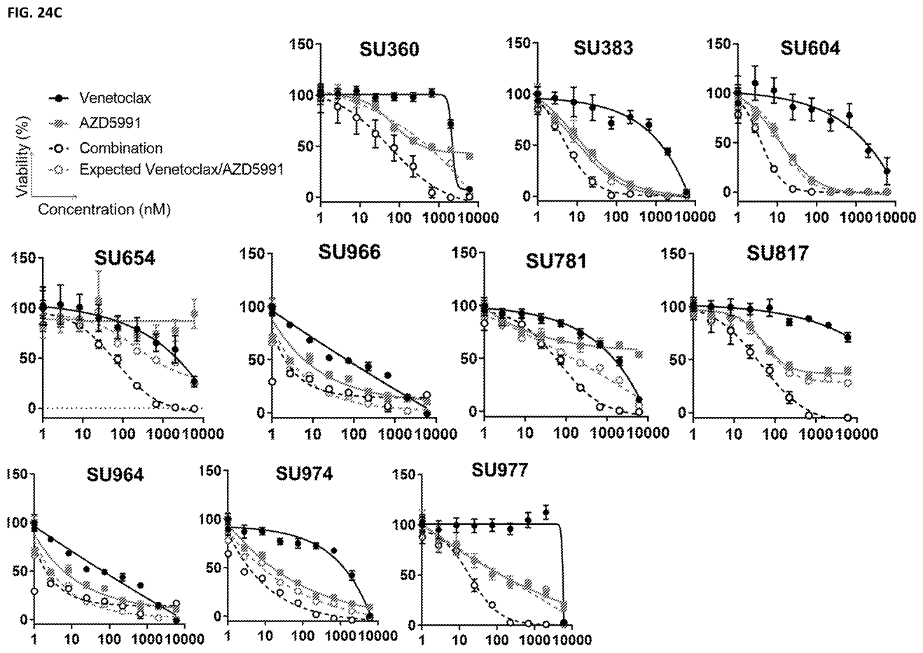

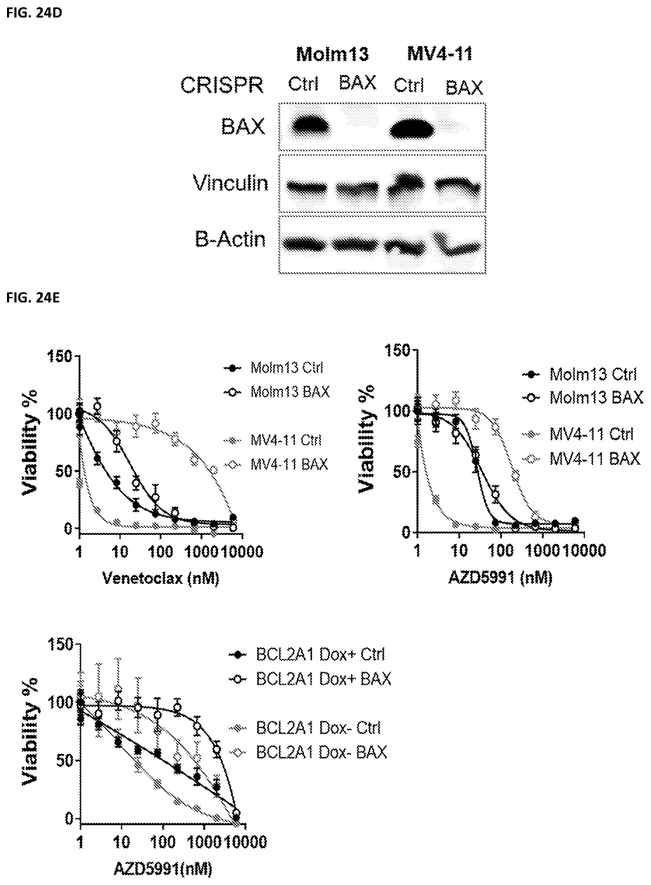

To understand the mechanisms associated with the robust synergy of venetoclax in combination with AZD5991, we performed BCL2 family protein immunoblotting. We observed that the combination uniquely generated a cleaved form of BAX even at low concentration (30 nM) (FIGS. 18J and 24D), which was previously shown to induce stronger apoptosis than the full-length form of BAX^{41,42}. Accordingly, knockout of BAX imparted resistance to the AZD5991 and venetoclax combination in multiple settings (FIGS. 18K-L and 24E), suggesting that the cleaved BAX may mediate the robust cytotoxic effect of the combination.

### Discussion

Previous studies have shown that venetoclax sensitivity is correlated and dependent on BCL2 expression, and upregulation of BCL-xL and MCL1 have been identified as major contributors to venetoclax resistance (9,19). Surprisingly, we observed from the Beat AML cohort that higher BCL2A1 expression demonstrated the strongest correlation with venetoclax resistance. We further validated that BCL2A1 upregulation mediated resistance to venetoclax-induced apoptosis in AML cell line models. Importantly, BCL2A1 overexpression also conferred relative resistance to common venetoclax combinations and other BCL2 targeted therapies. This is in agreement with a previous study showing BCL2A1 expression inversely correlated with the activity of ABT-737 in CLL⁴⁴. Notably, studies have also implicated that long-term treatment with BCL2 inhibitors and other chemotherapeutics can further upregulate the expression of BCL2A1, leading to BCL2 inhibitor resistance and chemoresistance^{45,46}. Therefore, there is an immediate unmet need for targeting BCL2A1 in AML. In the absence of a pharmacologic inhibitor of BCL2A1, we knocked down BCL2A1 with shRNAs, which inhibited proliferation and enhanced apoptosis of leukemia cells and synergized with venetoclax to inhibit AML cell survival.

Using clinically applicable markers to identify patients who are most likely to respond to a particular treatment is a critical step for future AML clinical management with the recent approval of an increasing number of novel therapies. In the current study, we also identified that AML with high monocyte/neutrophil count, FAB M4/M5, AM L with high levels of monocyte/neutrophil markers (CD14/CD369), and MDS/MPN transformed AML are relatively resistant to venetoclax. These phenotypic markers may correlate with increased expression of BCL2A1 in monocytic leukemia precursors and their progeny as we observed high BCL2A1 expression in M4/M5 and transformed AML, strong positive correlation between CD369/CD14 and BCL2A1, and specifically increased expression of BCL2A1 in the monocyte and granulocyte lineages during HSC differentiation. This is in line with previous studies which suggest that cell differentiation status affects venetoclax sensitivity in T cell lymphoblastic leukemia⁴⁷⁻⁴⁹. AML blast and PB counts and immunophenotyping are already routinely performed in clinical evaluation, therefore, blast and monocyte/neutrophil count, as well as CD14 expression, are ideal biomarker candidates to predict response to venetoclax-containing regimens. Unfortunately, CD369 is not included in most current immunophenotyping panels and FAB phenotype is less frequently assessed and reported in clinical practice. For predicting response to venetoclax-containing regimens, our data strongly support the continued evaluation of FAB phenotype as well as the addition of CD369 into immunophenotyping panels.

BCL2 family genes are rarely mutated in leukemia. However, a myriad of genetic changes accompanies the evolution of a normal cell to a cancer cell, partly impacting BCL2 family protein expression. KRAS mutations were previously shown to induce BCL-xL upregulation, which subsequently mediates treatment resistance in solid tumors^{50,51}. In the current study, we identified and validated that KRAS mutations conferred resistance to venetoclax monotherapy or combination treatment, probably through downregulating BAX and sustaining pMCL1, rather than through ERK pathway activation, since we observed upregulation of pERK by both NRAS and KRAS mutants, yet NRAS mutations did not induce venetoclax resistance. PTPN11 mutations were previously shown to upregulate MCL1 to accelerate MLL-AF9-mediated leukemogenesis⁵². They have also been shown to promote lung tumorigenesis in a transgenic mouse model by upregulating BCL-xL⁵³. Here, we determined that PTPN11 mutations conferred venetoclax resistance via sustaining BCL-w and pMCL1 during venetoclax treatment, which could be circumvented by BCL-w and MCL1 inhibition. Further studies are ongoing to unveil the detailed mechanisms of the deregulated BCL2 family pathway by KRAS and PTPN11 mutations in AML. Next-generation sequencing has become part of routine clinical practice in the treatment of AML, and KRAS and PTPN11 hotspot mutations are included in common AML genetic testing panels making them good candidates as biomarkers for venetoclax-containing regimens.

As we have established biomarkers for venetoclax resistance, we further explored potential therapeutics to overcome venetoclax resistance. We observed that venetoclax and AZD5991 combination could overcome resistance and demonstrated robust synergy across all three venetoclax resistance cell line models and primary AML patient samples in vitro and in vivo. We further uncovered evidence that this drug combination induced potent pro-apoptotic cleaved BAX that may account for the synergistic effects. It is unlikely that the cleaved BAX is the consequence rather than the cause of the massive apoptosis induced by the combination, since we did not observe the cleaved BAX even at high concentration of venetoclax or AZD5991 where apoptosis is evident as indicated by the cleaved PARP. However, how the combination induces BAX cleavage is unknown and a topic of our future study.

The encouraging efficacy of novel targeted therapies has brought about huge advances in the treatment of AML. However, patients exhibit heterogeneous responses and often develop resistance that limits long-term disease-free and overall survival. Establishing biomarkers to predict drug response and exploration of alternative therapeutics to overcome drug resistance are ongoing areas of investigation. By mining large leukemia functional genomic data sets from the Beat AML cohort, we have identified, validated, and revealed the mechanisms of clinically applicable biomarkers for venetoclax resistance in AML, and further determined that venetoclax in combination with the MCL1 inhibitor AZD5991 could overcome venetoclax resistance via cleavage of BAX.

Provided is a method of treating venetoclax-resistant Acute Myeloid Leukemia in a human subject, the method comprising administering to the human subject:
a) a pharmaceutically effective amount of venetoclax, or a pharmaceutically acceptable salt thereof; and
b) a pharmaceutically effective amount of AZD5991, or a pharmaceutically acceptable salt thereof.

### Materials and Methods

### Patient Samples

Samples were obtained with written, informed consents obtained from all patients according to the Declaration of Helsinki and approved Institutional Review Boards (IRB) protocol (Oregon Health & Science University (OHSU) IRB no. 4422; Stanford IRB no. 18329 and 6453). Mononuclear cells (MNCs) were isolated by Ficoll gradient centrifugation from freshly obtained BM aspirates or PB draws. Cells were frozen in liquid nitrogen for subsequent flow cytometry (FACS) analysis and inhibitor assay. Stanford primary AML sample information is summarized in Supplementary Data 1.

### Cell Cultures

HEK 293T/17 cells (provided by Dr. Richard Van Etten) were maintained in DMEM supplemented with 10% FBS, L-glutamine, and penicillin/streptomycin. Molm13, MV4-11, CTS, U937, K562, NB4, HEL, and THP-1 were maintained in RPMI 1640 (Invitrogen) supplemented with 10% FBS, L-glutamine, and penicillin/streptomycin. Mycoplasma contamination was routinely tested (once per month). Only mycoplasma free cells were used in the experiments. Authentication was performed on all cell lines used in this study at the OHSU DNA Services Core facility. Primary leukemia cells and cord blood cells were cultured in IMDM supplemented with 20% FBS, L-glutamine, IL-3 (50 ng/ml), IL-6 (25 ng/ml), FLT3 ligand (50 ng/ml), TPO (50 ng/ml) and SCF (100 ng/ml). Molm13 cells were obtained from Sanger Institute Cancer cell line panel; CTS cells were provided by Dr. Jude Fitzgibbon from Barts Cancer Institute UK, and all the other cell lines were purchased from ATCC. All cytokines were purchased from Peprotech.

### Mice

All animal studies were performed in accordance with protocols approved by Stanford Animal Care and Use Committees. Seven-week-old female NSG mice (#005557, The Jackson Lab) were irradiated with 200 rad and intravenously injected with AML cell line (BCL2A1 overexpressing MV4-11, 0.5 million per mouse) or CD3+ cell-depleted primary patient cells (SU176 harbors KRAS G12D, 3 million per mouse) as indicated in the schematic outlines for each mouse model (FIGS. 18H and 18K). After confirmation of engraftment, the mice were randomly assigned to four groups (n=3 per group) and treated with vehicle, venetolcax (#V-3579, LC Laboratories,100 mg/kg, oral gavage, per day), AZD5991 (#CT-A5991, Chemietek, 100 mg/kg intravenous, per week) or the combination. Venetoclax was prepared weekly in 0.5% Tween 80, 30% polyethyleneglycol-400, and 15% propylene glycol (18.7 mg/ml). AZD5991 was formulated weekly in DMSO (200 mg/ml). The leukemic burden of BCL2A1 overexpressing MV4-11 cells was monitored by bioluminescence imaging at indicated time points. Briefly, mice were anesthetized and injected intraperitoneally with firefly luciferase substrate D-luciferin and then imaged with the IVIS-100 in vivo imaging system (PerkinElmer). The leukemic burden of primary AML cells was monitored by FACS analysis of BM engraftment using anti-human-CD45 (#560367, BD Biosciences) and anti-mouse-CD45 (#355404, Biolegend,) at indicated time point or when the mice became moribund.

### Lentiviral shRNA Constructs

shRNAs targeting BCL2A1 were designed using siRNA Dharmacon Design Center and cloned into pRSI9 U6-sh-UbiC-TagRFP/GFP lentiviral vector. pRSI9 U6-sh-UbiC-TagGFP was cloned from pRSI9 U6-sh-UbiC-TagRFP (#28289, Addgene). ShS: ATCTCGCTTGGGCGAGAGTAAG; Sh1: GTTTGAAGACGGCATCAT; Sh2: TTTGTAGCACTCTGGACGT; Sh3: ACCTTCAAATGCAAATATG; and Sh3UTR: AATCGTTTCCATATCAGTC.

### Colony Forming Unit Assay (CFU) Assay

Mouse BM cells were harvest from WT BALB/c mice (#001026, The Jackson Lab). All mouse work was performed with approval from the OHSU Institutional Animal Care and Use Committee. For the BM transduction experiment, BM lineage negative cells were infected with retrovirus expressing KRAS or PTPN11 WT and mutants. Two thousand lineage negative cells per well were seeded into 6-well plate with 1.1 mL of methylcellulose medium (#03534, STEMCELL) for 10 days and the colony number was counted.

Fresh human umbilical cord blood cells were purchased from the New York Blood Center. MNCs from each sample were isolated by Ficoll separation, enriched for CD34 using CD34 magnetic beads (#17856, STEMCELL), transduced with lentivirus expressing sh1 or shS, and sorted by FACS. Two thousand cells were then seeded into 6-well plate with 1.1 mL of methylcellulose medium (#04434, STEMCELL) for 10 days and colony cells were counted.

### Virus Transduction

PTPN11, KRAS, and NRAS mutations were generated using the QuikChange II XL site-directed mutagenesis kit (Agilent Technologies) on the respective pENTR or pDonor WT vectors (#GC-Z2134, GeneCopoeia; #81923, Addgene; and #82151, Addgene respectively). Gblock of the full length of BCL2A1 were purchased from IDT and cloned into a pENTR vector via TOPO TA cloning. All entry vectors were then cloned into a gateway compatible MSCV-IRES-GFP (#20672, Addgene) or MSCV-IRES-mCherry (#52114, Addgene) retroviral vector or a Tet-inducible lentiviral vector, pInducer21 (Addgene, #46948) via Gateway Cloning System (Invitrogen). A sgRNA targeting BAX was cloned into lentiCRISPR v2 as previously described ²⁶.

Retrovirus was produced by transfecting HEK 293T/17 cells together with an EcoPac helper plasmid). Lentivirus was produced by transfecting HEK 293T/17 cells together with psPAX2 and VSVG. After 2 days, the virus-containing supernatants were filtered and infected to cells followed by FACS sorting.

### FACS

Cells were stained with antibodies for 20 minutes (min) at room temperature and washed twice with PBS. Membrane expression of CD45, CD369, and CD14 was analyzed by FACS Aria and Flowjo Software (Treestar, Ashland, Oreg.).

### Ex Vivo Functional Drug Screens

10,000 primary AML cells or 1250 leukemia cell line cells per well were seeded into 384-well plates containing does gradients of venetoclax and venetoclax combinations (equal concentration) and cultured for 3 days. Cell viability was measured using a methanethiosulfonate (MTS)-based assay and read at 490 nm after 1-12 hour (h) using a plate reader. Cell viability was determined by comparing the absorbance of drug-treated cells to that of untreated controls set at 100%. IC50 values were calculated by a regression curve fit analysis using GraphPad Prism8 software.

### Immunoblotting

An equal number of cells were pelleted and lysed with the RIPA Lysis buffer solution at 4° C. Supernatants were centrifuged and the protein concentration was quantified with BCA Protein Assay. An equal amount of whole-cell lysate was mixed with 3X SDS sample buffer (75 mM Tris (pH 6.8), 3% SDS, 15% glycerol, and 0.1% bromophenol blue) containing 8% β-mercaptoethanol. The mixed samples were boiled for 5 min in a 95° C. heat block before loading on a 4-15% Tris-HCl gradient gel. The proteins were transferred onto a nitrocellulose membrane and blocked with 5% BSA TBST buffer and incubated with antibodies against BCL2 family protein, PARP, Actin, Vinculin, etc., and HRP conjugated secondary antibodies against mouse IgG and rabbit IgG.

### Proliferation Assay

Leukemia cells, CD34+ cord blood cells or primary leukemia cells were transduced with shS, sh1 and/or sh3UTR. Cells were cultured and passed as normal and samples were taken for FACS analysis at day 3 and day 6. The percentage difference in growth rate between shS and sh1 cells was calculated based on the percentage change of GFP+ cells.

### Apoptosis Assay

The detection of apoptotic cells was performed by staining the cells with Annexin V/PI. Cells were stained with APC-labeled Annexin V and PI (#640928, Biolegend) according to the manufacturer's instructions and analyzed by FACS. Annexin V was used as an apoptotic cell marker, whereas propidium iodide (PI) was used as a necrotic cell marker.

### Real-Time PCR

Primary leukemia blasts were sorted into CD369/CD14+ and CD369/CD14− cell population using PE anti-CD369 (#355404, Biolegend) and APC-Cy7 anti-CD14 antibodies (#367108, Biolegend) by FACS. Total RNA was extracted using an RNA isolation kit according to the manufacturer's instructions. 200 ng of total RNA was used for cDNA synthesis using high capacity reverse transcription kit. Reactions were run in duplicate. Expression of apoptotic related genes was normalized to the geometric mean of housekeeping gene GAPDH or Actin to control the variability in expression levels and were analyzed using the 2 -ΔΔCT method. All samples within an experiment were reverse transcribed at the same time, the resulting cDNA diluted 1:5 in nuclease-free water and stored in aliquots at −80° C. until used. Primers are selected from a Human Apoptosis Primer Library (#HPA-I, Real Time Primers, LLC)

### Gene Expression Studies

Clinical information, mutation and gene expression data for inhibitor correlation analysis were obtained from the Beat AML and CNL public Vizome interface [www.vizome.org] and expressed as normalized RPKM. AML TCGA data were obtained from cBioPortal [https://www.cbioportal.org/] and expressed as log-transformed RNA Seq V2 RSEM. Data for CD369 and CD14 expression in a cohort of CML cases was mined from an internal dataset that is being prepared for publication and expressed as normalized RPKM. Figures for gene expression during normal hematopoiesis and in various leukemia were downloaded Bloodspot [http://servers.binf.ku.dk/bloodspot/].

### Evaluation of Combinatorial Effect of Combination Drugs

We used Excess over Bliss (EOB) independence model66 to quantify the synergy for crenolanib/trametinib combination at each drug concentration. EOB evaluates if the combined effect of two compounds is significantly greater or smaller than the combination of their individual (independent) effects and is measured by calculating the difference between the observed and predicted inhibition of the drug combination. For two single compounds with inhibition effects A and B, the predicted inhibition for the drug combination is calculated as C=A+B−A*B. The two-agent combination inhibition is defined as AB. The predicted combination viability of drug A and B combination is defined as (A+B−A*B)%. EOB can be calculated by Z=AB−C. Z Plus score (>0) indicates a synergistic effect, and Z minus score (<0) indicates an antagonistic effect.

### Quantification and Statistical Analysis

Statistical analysis was performed on GraphPad Prism software 8.0. The data were expressed as the mean±standard error of the mean (MED). Statistical significance was determined using two-tailed Student's t-tests (Mann-Whitney test), one-way ANOVA, or log-rank (Mantel-Cox) tests as indicated and expressed as p-values (*p<0.05, **p<0.01, ***p<0.001, and ****p<0.0001).

### REFERENCES

1. Papaemmanuil, E. et al. Genomic Classification and Prognosis in Acute Myeloid Leukemia. N. Engl. J. Med. 374, 2209-2221 (2016).
2. Patel, J. P. et al. Prognostic Relevance of Integrated Genetic Profiling in Acute Myeloid Leukemia. N. Engl. J. Med. (2012). doi:10.1056/NEJMoa1112304
3. Ley, T. J. et al. Genomic and epigenomic landscapes of adult de novo acute myeloid leukemia. N. Engl. J. Med. 368, 2059-74 (2013).
4. DiNardo, C. D. et al. Clinical experience with the BCL2-inhibitor venetoclax in combination therapy for relapsed and refractory acute myeloid leukemia and related myeloid malignancies. Am. J. Hematol. (2018). doi:10.1002/ajh.25000
5. Wei, A. et al. Phase 1/2 Study of Venetoclax with Low-Dose Cytarabine in Treatment-Naive, Elderly Patients with Acute Myeloid Leukemia Unfit for Intensive Chemotherapy: 1-Year Outcomes. Blood (2017).
6. Dinardo, C. D. et al. Durable response with venetoclax in combination with decitabine or azacitadine in elderly patients with acute myeloid leukemia (AML). J. Clin. Oncol. (2018). doi:10.1200/jco.2018.36.15_suppl.7010
7. Pollyea, D. A. et al. Venetoclax in Combination with Hypomethylating Agents Induces Rapid, Deep, and Durable Responses in Patients with AML Ineligible for Intensive Therapy. Blood (2018). doi:10.1182/blood-2018-99-117179
8. DiNardo, C. D. et al. Venetoclax combined with decitabine or azacitidine in treatment-naive, elderly patients with acute myeloid leukemia. Blood (2019). doi:10.1182/blood-2018-08-868752
9. Konopleva, M. et al. Efficacy and biological correlates of response in a phase II study of venetoclax monotherapy in patients with acute Myelogenous Leukemia. Cancer Discov. (2016). doi:10.1158/2159-8290.CD-16-0313
10. DiNardo, C. D. et al. Safety and preliminary efficacy of venetoclax with decitabine or azacitidine in elderly patients with previously untreated acute myeloid leukaemia: a non-randomised, open-label, phase 1b study. Lancet Oncol. (2018). doi:10.1016/S1470-2045(18)30010-X
11. Kaufmann, S. H. et al. Elevated expression of the apoptotic regulator Mcl-1 at the time of leukemic relapse. Blood (1998).
12. Karakas, T. et al. High expression of bcl-2 mRNA as a determinant of poor prognosis in acute myeloid leukemia. Ann. Oncol. (1998). doi:10.1023/A:1008255511404
13. Campos, L. et al. High expression of bcl-2 protein in acute myeloid leukemia cells is associated with poor response to chemotherapy. Blood 81, 3091-3096 (1993).
14. Stilgenbauer, S. et al. Venetoclax in relapsed or refractory chronic lymphocytic leukaemia with 17p deletion: a multicentre, open-label, phase 2 study. Lancet Oncol. (2016). doi:10.1016/S1470-2045(16)30019-5
15. Roberts, A. W. et al. Targeting BCL2 with Venetoclax in Relapsed Chronic Lymphocytic Leukemia. N. Engl. J. Med. (2015). doi:10.1056/nejmoa1513257
16. Pollyea, D. A. et al. Results of a phase 1b study of venetoclax plus decitabine or azacitidine in untreated acute myeloid leukemia patients ≥65 years ineligible for standard induction therapy. J. Clin. Oncol. (2018). doi:10.1200/jco.2016.34.15_suppl.7009
17. Jayappa, K. D. et al. Microenvironmental agonists generate de novo phenotypic resistance to combined ibrutinib plus venetoclax in CLL and MCL. Blood Adv. (2017).
18. Oppermann, S. et al. High-content screening identifies kinase inhibitors that overcome venetoclax resistance in activated CLL cells. Blood (2016). doi:10.1182/blood-2015-12-687814
19. Punnoose, E. A. et al. Expression Profile of BCL-2, BCL-XL, and MCL-1 Predicts Pharmacological Response to the BCL-2 Selective Antagonist Venetoclax in Multiple Myeloma Models. Mol. Cancer Ther. (2016). doi:10.1158/1535-7163.MCT-15-0730
20. Pan, R. et al. Synthetic Lethality of Combined Bcl-2 Inhibition and p53 Activation in AML: Mechanisms and Superior Antileukemic Efficacy. Cancer Cell (2017). doi:10.1016/j.ccell.2017.11.003
21. Ishizawa, J. et al. Mitochondrial ClpP-Mediated Proteolysis Induces Selective Cancer Cell Lethality. Cancer Cell (2019). doi:10.1016/j.ccell.2019.03.014
22. Kuntz, E. M. et al. Targeting mitochondrial oxidative phosphorylation eradicates therapy-resistant chronic myeloid leukemia stem cells. Nat. Med. (2017). doi:10.1038/nm.4399
23. Bajpai, R. et al. Targeting glutamine metabolism in multiple myeloma enhances BIM binding to BCL-2 eliciting synthetic lethality to venetoclax. Oncogene (2016). doi:10.1038/onc.2015.464
24. Chen, X. et al. Targeting mitochondrial structure sensitizes acute myeloid leukemia to Venetoclax treatment. Cancer Discov. (2019). doi:10.1158/2159-8290.CD-19-0117
25. Stevens, B. M. et al. PTPN11 Mutations Confer Unique Metabolic Properties and Increase Resistance to Venetoclax and Azacitidine in Acute Myelogenous Leukemia. Blood (2018). doi:10.1182/BLOOD-2018-99-119806
26. Nechiporuk, T. et al. The TP53 Apoptotic Network is a Primary Mediator of Resistance to BCL2 inhibition in AML Cells. Cancer Discov. (2019). doi:10.1158/2159-8290.CD-19-0125
27. Han, L. et al. Targeting MAPK Signaling Pathway with Cobimetinib (GDC-0973) Enhances Anti-Leukemia Efficacy of Venetoclax (ABT-199/GDC-0199) in Acute Myeloid Leukemia Models. Clin. Lymphoma Myeloma Leuk. (2017). doi:10.1016/j.clml.2017.07.064
28. Pham, L. V. et al. Strategic therapeutic targeting to overcome venetoclax resistance in aggressive B-cell lymphomas. Clin. Cancer Res. (2018). doi:10.1158/1078-0432.CCR-17-3004
29. Tyner, J. W. et al. Functional genomic landscape of acute myeloid leukaemia. Nature 562, 526-531 (2018).
30. Bak, R. O. et al. Multiplexed genetic engineering of human hematopoietic stem and progenitor cells using CRISPR/Cas9 and AAV6. Elife 6, e27873 (2017).
31. Sochalska, M. et al. Conditional knockdown of BCL2A1 reveals rate-limiting roles in BCR-dependent B-cell survival. Cell Death Differ. (2016). doi:10.1038/cdd.2015.130
32. Zhang, H. et al. Genomic landscape of Neutrophilic Leukemias of Ambiguous Diagnosis. Blood (2019). doi:10.1182/blood.2019000611
33. Domina, A. M., Vrana, J. A., Gregory, M. A., Hann, S. R. & Craig, R. W. MCL1 is phosphorylated in the PEST region and stabilized upon ERK activation in viable cells, and at additional sites with cytotoxic okadaic acid or taxol. Oncogene (2004). doi:10.1038/sj.onc.1207692
34. Mazumder, S., Choudhary, G. S., Al-harbi, S. & Almasan, A. Mcl-1 phosphorylation defines ABT-737 resistance that can be overcome by increased NOXA expression in leukemic B cells. Cancer Res. (2012). doi:10.1158/0008-5472.CAN-11-4106
35. Yin, C., Knudson, C. M., Korsmeyer, S. J. & Van Dyke, T. Bax suppresses tumorigenesis and stimulates apoptosis in vivo. Nature (1997). doi:10.1038/385637a0
36. Lee, T. et al. Discovery of Potent Myeloid Cell Leukemia-1 (Mcl-1) Inhibitors That Demonstrate in Vivo Activity in Mouse Xenograft Models of Human Cancer. J. Med. Chem. 62, 3971-3988 (2019).
37. Li, Z., He, S. & Look, A. T. The MCL1-specific inhibitor S63845 acts synergistically with venetoclax/ABT-199 to induce apoptosis in T-cell acute lymphoblastic leukemia cells. Leukemia (2019). doi:10.1038/s41375-018-0201-2
38. Moujalled, D. M. et al. Combining BH3-mimetics to target both BCL-2 and MCL1 has potent activity in pre-clinical models of acute myeloid leukemia. Leukemia (2019). doi:10.1038/s41375-018-0261-3
39. Wattel, E. et al. p53 mutations are associated with resistance to chemotherapy and short survival in hematologic malignancies. Blood (1994).
40. Zhang, H. et al. Clinical resistance to crenolanib in acute myeloid leukemia due to diverse molecular mechanisms. Nat. Commun. (2019). doi:10.1038/s41467-018-08263-x
41. Gao, G. & Dou, Q. P. N-terminal cleavage of Bax by calpain generates a potent proapoptotic 18-kDa fragment that promotes Bcl-2-independent cytochrome C release and apoptotic cell death. J. Cell. Biochem. (2000). doi:10.1002/1097-4644(20010101)80:1<53::AID-JCB60>3.0.CO;2-E
42. Wood, D. E. & Newcomb, E. W. Cleavage of bax enhances its cell death function. Exp. Cell Res. (2000). doi:10.1006/excr.2000.4859
43. Phillips, D. C. et al. Loss in MCL-1 function sensitizes non-Hodgkin's lymphoma cell lines to the BCL-2-selective inhibitor venetoclax (ABT-199). Blood Cancer J. (2015). doi:10.1038/bcj.2015.88
44. Yecies, D., Carlson, N. E., Deng, J. & Letai, A. Acquired resistance to ABT-737 in lymphoma cells that up-regulate MCL-1 and BFL-1. Blood (2010). doi:10.1182/blood-2009-07-233304
45. Cheng, Q., Lee, H. H., Li, Y., Parks, T. P. & Cheng, G. Upregulation of Bcl-x an Bfl-1 as a potential mechanism of chemoresistance, which can be overcome by NE-κB inhibition. Oncogene (2000). doi:10.1038/sj.onc.1203861
46. Rasooly, R. et al. Retinoid X Receptor Agonists Increase Bcl2a1 Expression and Decrease Apoptosis of Naive T Lymphocytes. J. Immunol. (2014). doi:10.4049/jimmunol.175.12.7916
47. Anderson, N. M. et al. BCL2-specific inhibitor ABT-199 synergizes strongly with cytarabine against the early immature LOUCY cell line but not more-differentiated T-ALL cell lines. Leukemia (2014). doi:10.1038/leu.2013.377
48. Ni Chonghaile, T. et al. Maturation stage of T-cell acute lymphoblastic leukemia determines BCL-2 versus BCL-XL dependence and sensitivity to ABT-199. Cancer Discov. (2014). doi:10.1158/2159-8290.CD-14-0353
49. Peirs, S. et al. ABT-199 mediated inhibition of BCL-2 as a novel therapeutic strategy in T-cell acute lymphoblastic leukemia. Blood (2014). doi:10.1182/blood-2014-05-574566
50. Zaanan, A. et al. The mutant KRAS gene up-regulates BCL-XL protein via STAT3 to confer apoptosis resistance that is reversed by BIM protein induction and BCL-XL antagonism. J. Biol. Chem. (2015). doi:10.1074/jbc.M115.657833
51. Corcoran, R. B. et al. Synthetic Lethal Interaction of Combined BCL-XL and MEK Inhibition Promotes Tumor Regressions in KRAS Mutant Cancer Models. Cancer Cell (2013). doi:10.1016/j.ccr.2012.11.007
52. Chen, L. et al. Mutated Ptpn11 alters leukemic stem cell frequency and reduces the sensitivity of acute myeloid leukemia cells to Mcl1 inhibition. Leukemia (2015). doi:10.1038/leu.2015.18
53. Lee, Y. H., et al. Angiotensin-II-induced apoptosis requires regulation of nucleolin and Bcl-xL by SHP-2 in primary lung endothelial cells. J. Cell Sci. (2010). doi:10.1242/jcs.063545

What is claimed:

1. A method of treating venetoclax-resistant Acute Myeloid Leukemia in a human subject, the method comprising administering to the human subject:
c) a pharmaceutically effective amount of venetoclax, or a pharmaceutically acceptable salt thereof; and
d) a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

2. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of palbociclib, or a pharmaceutically acceptable salt thereof.

3. The method of claim 1, wherein administered to the human subject in need thereof is a pharmaceutically effective amount of venetoclax, or a pharmaceutically acceptable salt thereof, and a pharmaceutically effective amount of at least two therapeutic agents selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.

4. The method of claim 1, wherein venetoclax is administered to the human subject in need thereof at a dose of from about 10 mg to 500 mg once or twice daily.

5. The method of claim 1, wherein venetoclax is administered to the human subject in need thereof at a dose of from about 50 mg to 400 mg once or twice daily.

6. The method of claim 2, wherein palbociclib is administered to the human subject in need thereof at a dose of from about 50 mg per day to about 300 mg per day.

7. The method of claim 2, wherein palbociclib is administered to the human subject in need thereof at a dose of from about 50 mg per day to about 200 mg per day.

8. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of ARRY-382, or a pharmaceutically acceptable salt thereof.

10. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of sorafenib, or a pharmaceutically acceptable salt thereof.

11. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of ruxolitinib, or a pharmaceutically acceptable salt thereof.

12. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of dasitinib, or a pharmaceutically acceptable salt thereof.

13. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of doramapimod, or a pharmaceutically acceptable salt thereof.

14. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of quizartinib, or a pharmaceutically acceptable salt thereof.

15. The method of claim 1 of treating venetoclax-resistant Acute Myeloid Leukemia in a human subject, wherein the method comprises administering to the human subject:
a) a pharmaceutically effective amount of venetoclax, or a pharmaceutically acceptable salt thereof; and
b) a pharmaceutically effective amount of idelalisib, or a pharmaceutically acceptable salt thereof.

16. A method of diagnosing and treating venetoclax-resistant Acute Myeloid Leukemia in a human subject, the method comprising:
a) obtaining a biological sample from the human subject;
b) detecting whether one or more mutations selected from the group of a TET2 mutation, a KRAS mutation, a PTPN11 mutation, and a SF3B1 mutation is present in the biological sample;
c) diagnosing the human subject with venetoclax-resistant Acute Myeloid Leukemia when the presence of one or more mutations selected from the group of a TET2 mutation, a KRAS mutation, a PTPN11 mutation, and a SF3B1 mutation is detected in the biological sample; and
d) administering to the human subject in need thereof a pharmaceutically effective amount of venetoclax, or a pharmaceutically acceptable salt thereof, and a pharmaceutically effective amount of palbociclib, or a pharmaceutically acceptable salt thereof.

17. The method of claim 16, wherein the KRAS mutation is a KRAS G12D mutation.

18. The method of claim 16, wherein the PTPN11 mutation is a PTPN11 A72D mutation.

19. A method of diagnosing and treating venetoclax-resistant Acute Myeloid Leukemia in a human subject, the method comprising:
a) obtaining a biological sample from the human subject;
b) detecting whether a high level of expression of CLEC7A (CD369), BCL2A1, or both of CLEC7A (CD369) and BCL2A1 is present in the biological sample;
c) diagnosing the human subject with venetoclax-resistant Acute Myeloid Leukemia when the presence of a high level of expression of CLEC7A (CD369), BCL2A1, or both of CLEC7A (CD369) and BCL2A1 is detected in the biological sample; and
d) administering to the human subject in need thereof a pharmaceutically effective amount of venetoclax, or a pharmaceutically acceptable salt thereof; and a pharmaceutically effective amount of a second therapeutic agent selected from the group of palbociclib, ARRY-382 (Array 382), sorafenib, ruxolitinib, dasatinib, doramapimod (BIRB 796), quizartinib, and idelalisib, or a pharmaceutically acceptable salt thereof.
